SEQUENCE LISTING (114 sequences)

SEQ ID NO: 1 - protein, 125 aa, synthetic construct
EVQLVESGAEVKKPGESLKISCKASGYSFTRFYIGWVRQMPGKGLEWMGLIYPGDSDTKYSPSFQGQVTISVDKSINTAYLQWNSLKASDTAMYYCARHIRYFDWFSHYYYMDVWGKGTMVTVSS

SEQ ID NO: 2 - protein, 5 aa, synthetic construct
RFYIG

SEQ ID NO: 3 - protein, 17 aa, synthetic construct
LIYPGDSDTKYSPSFQG

SEQ ID NO: 4 - protein, 16 aa, synthetic construct
HIRYFDWFSHYYYMDV

SEQ ID NO: 5 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 6 - protein, 6 aa, synthetic construct
QSISSY

SEQ ID NO: 7 - skipped

SEQ ID NO: 8 - protein, 9 aa, synthetic construct
QQSYSTPPT

SEQ ID NO: 9 - protein, 107 aa, synthetic construct [sequence omitted]

SEQ ID NO: 10 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 95 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 12 - protein, 107 aa, synthetic construct; identical to SEQ ID NO: 5

SEQ ID NO: 13 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 14 - protein, 6 aa, synthetic construct
QSISSY

SEQ ID NO: 15 - skipped

SEQ ID NO: 16 - protein, 10 aa, synthetic construct
QQSYSTPPIT

SEQ ID NO: 17 - protein, 95 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 18 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 19 - protein, 6 aa, synthetic construct
QSVSSN

SEQ ID NO: 20 - skipped

SEQ ID NO: 21 - protein, 9 aa, synthetic construct
QQYNNWPWT

SEQ ID NO: 22 - protein, 96 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 23 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 24 - protein, 7 aa, synthetic construct
QSVSSSY

SEQ ID NO: 25 - skipped

SEQ ID NO: 26 - protein, 9 aa, synthetic construct
QQYGSSPWT

SEQ ID NO: 27 - protein, 96 aa, synthetic construct
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 28 - protein, 108 aa, synthetic construct
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 29 - protein, 6 aa, synthetic construct
NIGRKS

SEQ ID NO: 30 - skipped

SEQ ID NO: 31 - protein, 11 aa, synthetic construct
QVWDGSSDHWV

SEQ ID NO: 32 - protein, 15 aa, synthetic construct
EPKSCDKTHTCPPCP

SEQ ID NO: 33 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 34 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 35 - protein, 110 aa, synthetic construct
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 36 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 454 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 454 aa, synthetic construct
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 456 aa, synthetic construct
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 41 - protein, 107 aa, synthetic construct [sequence omitted]

SEQ ID NO: 42 - protein, 107 aa, synthetic construct [sequence omitted]

SEQ ID NO: 43 - protein, 109 aa, synthetic construct [sequence omitted]

SEQ ID NO: 44 - protein, 112 aa, synthetic construct
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 45 - protein, 110 aa, synthetic construct
QAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLEIK

SEQ ID NO: 46 - protein, 107 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 47 - protein, 107 aa, synthetic construct [sequence omitted]

SEQ ID NO: 48 - protein, 112 aa, synthetic construct [sequence omitted]

SEQ ID NO: 49 - protein, 108 aa, synthetic construct
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 50 - protein, 112 aa, synthetic construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIK

SEQ ID NO: 51 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQMHPRAPKTFGQGTKVEIK

SEQ ID NO: 52 - protein, 113 aa, synthetic construct
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 53 - protein, 112 aa, synthetic construct
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 54 - protein, 107 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 55 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 56 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 57 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIK

SEQ ID NO: 58 - protein, 107 aa, synthetic construct
EIVMTQSPATLSVSPGERVSLSCRASQSISDYLHWYQQKSHESPRLLIKYASQSISGIPARFSGSGSGSEFTLTINSVEPEDVGVYYCQNGHSFPLTFGQGTKLEIK

SEQ ID NO: 59 - protein, 112 aa, synthetic construct
DIVMTQAAFSNPVTLGTSASISCRSSKSLLHSNGITYFFWYLQKPGLSPQLLIYQMSNLASGVPDRFSSSGSGTDFTLRISRVEAEDVGVYYCAQNLELPPTFGGGTKLEIK

SEQ ID NO: 60 - protein, 112 aa, synthetic construct
DVVMTQSPLSLPVTLGQPASISCKSSQSLLDSDGKTFLNWFQQRPGQSPRRLIYLVSKLDSGVPDRFSGSGSGTDFTLKISRVEAEDVGVYYCWQGTHFPYTFGQGTRLEIK

SEQ ID NO: 61 - protein, 106 aa, synthetic construct [sequence omitted]

SEQ ID NO: 62 - protein, 113 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 63 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 64 - protein, 108 aa, synthetic construct
DIQMTQSPSSLSASVGDRVSITCRPSQSFSRYINWYQQKPGKAPKLLIHAASSLVGGVPSRFSGSGSGTDFTLTISSLQPEDFATYYCQQTYSNPPITFGQGTRLEIK

SEQ ID NO: 65 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFVTYYCQQYNSYPRTFGQGTKVEIK

SEQ ID NO: 66 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 67 - protein, 106 aa, synthetic construct [sequence omitted]

SEQ ID NO: 68 - protein, 109 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIK

SEQ ID NO: 69 - protein, 110 aa, synthetic construct
QAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVL

SEQ ID NO: 70 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 71 - protein, 112 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 72 - protein, 108 aa, synthetic construct
DIQMTQSPSSLSASVGDRVSITCRPSQSFSRYINWYQQKPGKAPKLLIHAASSLVGGVPSRFSGSGSGTDFTLTISSLQPEDFATYYCQQTYSNPPITFGQGTRLEMK

SEQ ID NO: 73 - protein, 6 aa, synthetic construct
QDVNTA

SEQ ID NO: 74 - skipped

SEQ ID NO: 75 - protein, 9 aa, synthetic construct
QQHYTTPPT

SEQ ID NO: 76 - protein, 6 aa, synthetic construct
QSVSSY

SEQ ID NO: 77 - skipped

SEQ ID NO: 78 - protein, 9 aa, synthetic construct
QQSSNWPRT

SEQ ID NO: 79 - protein, 6 aa, synthetic construct
QSVSSY

SEQ ID NO: 80 - skipped

SEQ ID NO: 81 - protein, 11 aa, synthetic construct
QQRSNWPPALT

SEQ ID NO: 82 - protein, 13 aa, synthetic construct
QQRSNWPPALTFC

SEQ ID NO: 83 - protein, 9 aa, synthetic construct
SGSVTSDNF

SEQ ID NO: 84 - skipped

SEQ ID NO: 85 - protein, 10 aa, synthetic construct
ALFISNPSVE

SEQ ID NO: 86 - protein, 7 aa, synthetic construct
QSLGSSY

SEQ ID NO: 87 - skipped

SEQ ID NO: 88 - protein, 9 aa, synthetic construct
QQYADSPIT

SEQ ID NO: 89 - protein, 11 aa, synthetic construct
QSLANSYGNTF

SEQ ID NO: 90 - skipped

SEQ ID NO: 91 - protein, 9 aa, synthetic construct
LQGTHQPYT

SEQ ID NO: 92 - protein, 12 aa, synthetic construct
QSVLYAGNNRNY

SEQ ID NO: 93 - skipped

SEQ ID NO: 94 - protein, 9 aa, synthetic construct
QQYYTPPLA

SEQ ID NO: 95 - protein, 11 aa, synthetic construct
QSIVYSNGNTY

SEQ ID NO: 96 - skipped

SEQ ID NO: 97 - protein, 9 aa, synthetic construct
FQGSHVPYT

SEQ ID NO: 98 - protein, 6 aa, synthetic construct
QSVSSY

SEQ ID NO: 99 - skipped

SEQ ID NO: 100 - protein, 9 aa, synthetic construct
QQRSNWPIT

SEQ ID NO: 101 - protein, 6 aa, synthetic construct
QSVSSY

SEQ ID NO: 102 - skipped

SEQ ID NO: 103 - protein, 9 aa, synthetic construct
QQRSNWPPA

SEQ ID NO: 104 - protein, 6 aa, synthetic construct
QSISDY

SEQ ID NO: 105 - skipped

SEQ ID NO: 106 - protein, 9 aa, synthetic construct
QNGHSFPLT

SEQ ID NO: 107 - protein, 11 aa, synthetic construct
KSLLHSNGITY

SEQ ID NO: 108 - skipped

SEQ ID NO: 109 - protein, 9 aa, synthetic construct
AQNLELPPT

SEQ ID NO: 110 - protein, 6 aa, synthetic construct
QSFSRY

SEQ ID NO: 111 - skipped

SEQ ID NO: 112 - protein, 10 aa, synthetic construct
QQTYSNPPIT

SEQ ID NO: 113 - protein, 100 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELD

SEQ ID NO: 114 - protein, 126 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

(19) United States
(12) Patent Application Publication
(10) Pub. No.: US 2024/0262911 A1
(43) Pub. Date: Aug. 8, 2024

(54) PROMISCUOUS CD3 BINDING MOLECULES

(71) Applicant: Merus N.V., Utrecht (NL)

(72) Inventors: Cornelis Adriaan DE KRUIF, Utrecht (NL); Linda Johanna Aleida HENDRIKS, Utrecht (NL)

(21) Appl. No.: 18/398,935

(22) Filed: Dec. 28, 2023

(30) Foreign Application Priority Data
Dec. 30, 2022 (NL) 2033876

(51) Int. Cl. C07K 16/28; A61K 39/00

(52) U.S. Cl. CPC C07K 16/2809; A61K 2039/505; C07K 2317/31; C07K 2317/53; C07K 2317/565

(57) ABSTRACT

The present disclosure relates to the field of antibodies. In particular it relates to the field of producing therapeutic antibodies. More particularly it relates to anti-CD3 heavy chain variable regions that can pair with multiple different light chain variable regions to form functional CD3 binding domains, and CD3 binding domains and CD3 binding moieties comprising such anti-CD3 heavy chain variable regions.

### TECHNICAL FIELD

The present disclosure relates to the field of antibodies. In particular it relates to the field of producing therapeutic antibodies. More particularly it relates to anti-CD3 heavy chain variable regions (also referred to herein as CD3 heavy chain variable regions) that can pair with multiple different light chain variable regions to form functional CD3 binding domains, and CD3 binding domains and CD3 binding moieties comprising such CD3 heavy chain variable regions.

### BACKGROUND

Monoclonal antibodies that bind to human CD3 were among the first antibodies developed for therapeutic use in humans. Monoclonal CD3 binding antibodies are typically used for their immune suppressive qualities, for instance in transplant rejection. Antibodies which are multispecific for CD3 on T cells and for at least one surface target antigen on cancer cells, are capable of connecting any kind of T cell to a cancer cell, independently of T-cell receptor specificity, co-stimulation, or peptide antigen presentation. Such multispecific T-cell engaging antibodies show great promise in the treatment of various cancers and neoplastic growths.

It is an object of the present disclosure to provide CD3 heavy chain variable regions that can pair with multiple different light chain variable regions to form functional CD3 binding domains, i.e. to provide promiscuous CD3 heavy chain variable regions. Such CD3 heavy chain variable regions are particularly useful for the generation of multispecific T-cell engaging antibodies. They have a high likeliness of successfully being combined with the light chain variable region of any binding domain generated against any tumor-associated antigen (TAA). Multispecific antibodies comprising such CD3 binding domains and TAA binding domains can therefore efficiently be produced in a single host cell as common light chain antibodies. Further, promiscuous CD3 heavy chain variable regions can be readily combined with the light chain variable region of a clinically successful antibody. A multispecific antibody comprising such CD3 binding domain and binding domain of a clinically successful antibody has for instance the potential to extend the clinical success to other therapeutic areas, provide increased tumor specificity and thus safety, and/or be more effective in eradicating tumor cells.

### SUMMARY

In certain embodiments, the present disclosure provides a polypeptide comprising a heavy chain CDR3 (HCDR3) having an amino acid sequence as set forth in SEQ ID NO: 4; or having at least 70% sequence identity thereto.

In certain embodiments, the present disclosure provides a CD3 binding domain comprising the polypeptide as described herein.

In certain embodiments, the present disclosure provides a group of antigen-binding proteins that bind human CD3, wherein each antigen-binding protein within the group comprises a heavy chain variable region and light chain variable region, the heavy chain variable region comprising a HCDR3 having at least 70% sequence identity to the amino acid sequence as set forth in SEQ ID NO: 4.

In certain embodiments, the present disclosure provides a binding moiety comprising a polypeptide as described herein, or a CD3 binding domain as described herein.

In certain embodiments, the present disclosure provides a pharmaceutical composition comprising an effective amount of a polypeptide as described herein, or a CD3 binding domain as described herein, or a binding moiety as described herein, and a pharmaceutically acceptable carrier.

In certain embodiments, the present disclosure provides a polypeptide as described herein, or a CD3 binding domain as described herein, or a binding moiety as described herein, or a pharmaceutical composition as described herein, for use in therapy.

In certain embodiments, the present disclosure provides a polypeptide as described herein, or a CD3 binding domain as described herein, or a binding moiety as described herein, or a pharmaceutical composition as described herein, for use in the treatment of cancer.

In certain embodiments, the present disclosure provides a method for treating a disease, comprising administering an effective amount of a polypeptide as described herein, or a CD3 binding domain as described herein, or a binding moiety as described herein, or a pharmaceutical composition as described herein, to an individual in need thereof.

In certain embodiments, the present disclosure provides a method for treating cancer, comprising administering an effective amount of a polypeptide as described herein, or a CD3 binding domain as described herein, or a binding moiety as described herein, or a pharmaceutical composition as described herein, to an individual in need thereof.

In certain embodiments, the present disclosure provides a nucleic acid comprising a sequence encoding the polypeptide as described herein.

In certain embodiments, the present disclosure provides a vector comprising a nucleic acid as described herein.

In certain embodiments, the present disclosure provides a cell comprising a nucleic acid as described herein.

In certain embodiments, the present disclosure provides a cell producing a polypeptide as described herein, or a CD3 binding domain as described herein, or a binding moiety as described herein.

### DETAILED DESCRIPTION

One of the objects of the present disclosure is to provide novel CD3 heavy chain variable regions useful for the generation of therapeutic antibodies, in particular for the generation of T cell engaging antibodies. This object is met by the provision of several polypeptides, as well as binding domains and binding moieties comprising these polypeptides.

In certain embodiments, the polypeptides of the present disclosure are heavy chain variable regions that can pair with multiple different light chain variable regions and form functional binding domains that bind CD3. This property of the heavy chain variable regions is referred to in the art as promiscuity. The present inventors have identified an anti-CD3 heavy chain variable region, and a group comprising related anti-CD3 heavy chain variable regions, that is particularly promiscuous. These CD3 heavy chain variable regions have a high likeliness of successfully being combined with the light chain variable region of any binding domain generated against any tumor-associated antigen (TAA). Multispecific antibodies comprising such CD3 binding domains and TAA binding domains can therefore efficiently be produced in a single host cell as common light chain antibodies. Furthermore, the present inventors have shown that the promiscuous CD3 heavy chain variable regions of the present disclosure can be readily combined with the light chain variable region of several clinically successful antibodies. A multispecific antibody comprising such CD3 binding domain and binding domain of a clinically successful antibody has for instance the potential to extend the clinical success to other therapeutic areas, provide increased tumor specificity and thus safety, and/or be more effective in eradicating tumor cells. The CD3 heavy chain variable regions of the present disclosure are thus particularly useful for the generation of multispecific T cell engaging antibodies.

In certain embodiments, the present disclosure provides a polypeptide comprising a heavy chain CDR3 (HCDR3) having an amino acid sequence as set forth in SEQ ID NO: 4, or having at least 70% sequence identity thereto. In certain embodiments, the present disclosure provides a polypeptide comprising a heavy chain CDR3 (HCDR3) having an amino acid sequence as set forth in SEQ ID NO: 4, or having at least 70% sequence identity thereto and having the same length as SEQ ID NO:4.

In certain embodiments, the polypeptide is an immunoglobulin heavy chain or antigen-binding part thereof. In certain embodiments, the polypeptide is an immunoglobulin heavy chain, or antigen-binding part thereof, that, when combined with a suitable light chain, or antigen-binding part thereof, binds to CD3. For example, the antigen-binding part of an immunoglobulin heavy chain can be a heavy chain variable region with a CH1 region, or a heavy chain variable region. The antigen-binding part of a light chain can, for example, be a light chain variable region. In certain embodiments, said light chain is an immunoglobulin light chain.

In certain embodiments, the polypeptide when combined with a suitable light chain, or antigen-binding part thereof, binds to human CD3. The amino acid sequence of the signal peptide and extracellular domain of human CD38 is provided herein as SEQ ID NO: 113 and the amino acid sequence of the signal peptide and extracellular domain of human CD3& is provided herein as SEQ ID NO: 114. Binding to human CD3 can be determined in an ELISA assay by comparison with the background signal and/or a negative control. For example, a polypeptide, binding domain, antigen-binding protein, or binding moiety binds human CD3 when it has at least a two-fold higher binding signal than the background signal, and/or at least a two-fold higher binding activity than a negative control.

Antigen binding can be expressed in terms of specificity and affinity. The specificity determines which antigen or epitope thereof is specifically bound by a binding domain or binding moiety. The affinity is a measure for the strength of binding to a particular antigen or epitope.

The present inventors have identified a group of binding domains, which binding domains comprise a polypeptide, in particular a heavy chain variable region, comprising a HCDR3 that has at least 70% sequence identity to the amino acid sequence as set forth in SEQ ID NO: 4. In certain embodiments, such polypeptides, or binding domains or binding moieties comprising such polypeptides, are grouped based on the at least 70% sequence identity in the HCDR3. In certain embodiments, such polypeptides, or binding domains or binding moieties comprising such polypeptides, are grouped based on the at least 70% sequence identity in the HCDR3 and having the same HCDR3 length. In certain embodiments, such polypeptides, or binding domains or binding moieties comprising such polypeptides, are grouped based on the at least 70% sequence identity in the HCDR3, having the same HCDR3 length, and being derived from the same heavy chain variable region V gene segment.

“Percent (%) identity” as referring to nucleic acid or amino acid sequences herein is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more 15 nucleic acids/bases or amino acids. The alignment may also be carried out over individual CDR sequences. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percentage sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program NEEDLE. For the purpose of this disclosure, the NEEDLE program from the EMBOSS package is used to determine percent identity of amino acid and nucleic acid sequences (version 2.8.0, EMBOSS: The European Molecular Biology Open Software Suite (2000) Rice, P. Longden J. and Bleasby, A. Trends in Genetics 16, (6) pp 276-277, http://emboss.bioinformatics.nl/). For protein sequences, EBLOSUM62 is used for the substitution matrix. For DNA sequences, DNAFULL is used. The parameters used are a gap-open penalty of 10 and a gap extension penalty of 0.5.

After alignment by the program NEEDLE as described above the percentage of sequence identity between a query sequence and a sequence of this disclosure is calculated as follows: number of corresponding positions in the alignment showing an identical amino acid or identical nucleotide in both sequences divided by the total length of the alignment after subtraction of the total number of gaps in the alignment.

In certain embodiments, a polypeptide of the present disclosure comprises a heavy chain CDR1 (HCDR1) having an amino acid sequence as set forth in SEQ ID NO: 2, a heavy chain CDR2 (HCDR2) having an amino acid sequence as set forth in SEQ ID NO: 3, and a heavy chain CDR3 (HCDR3) having an amino acid sequence as set forth in SEQ ID NO: 4; wherein each of the HCDRs may comprise one, two, or three amino acid variations.

In certain embodiments, a polypeptide of the present disclosure comprises a heavy chain CDR1 (HCDR1) having an amino acid sequence as set forth in SEQ ID NO: 2, a heavy chain CDR2 (HCDR2) having an amino acid sequence as set forth in SEQ ID NO: 3, and a heavy chain CDR3 (HCDR3) having an amino acid sequence as set forth in SEQ ID NO: 4; wherein each of the HCDRs may contain one, two, or at most three amino acid variations. In certain embodiments, a polypeptide of the present disclosure comprises a heavy chain CDR1 (HCDR1) having an amino acid sequence as set forth in SEQ ID NO: 2, a heavy chain CDR2 (HCDR2) having an amino acid sequence as set forth in SEQ ID NO: 3, and a heavy chain CDR3 (HCDR3) having an amino acid sequence as set forth in SEQ ID NO: 4; wherein two of the HCDRs may contain one, two, or at most three amino acid variations. In certain embodiments, said two HCDRs are HCDR1 and HCDR2. In certain embodiments, a polypeptide of the present disclosure comprises a heavy chain CDR1 (HCDR1) having an amino acid sequence as set forth in SEQ ID NO: 2, a heavy chain CDR2 (HCDR2) having an amino acid sequence as set forth in SEQ ID NO: 3, and a heavy chain CDR3 (HCDR3) having an amino acid sequence as set forth in SEQ ID NO: 4; wherein one of the HCDRs may contain one, two, or at most three amino acid variations. In certain embodiments, said one HCDR is HCDR1 or HCDR2.

The heavy chain variable regions of a polypeptide of the present disclosure may comprise a limited number, such as for instance one, two, three, four, five, six, seven, eight, nine, or ten, non-conservative amino acid substitutions, or an unlimited number of conservative amino acid substitutions.

In certain embodiments, a polypeptide of the present disclosure also includes variants thereof, wherein each of the HCDRs may comprise one, two or three amino acid variations. In certain embodiments, only one or two HCDRs may comprise one, two, or three non-conservative amino acid variations. In certain embodiments, such variants do not comprise amino acid variations in HCDR3. In certain embodiments, the amino acid variation is a conservative amino acid substitution.

Typically, a conservative amino acid substitution involves a variation of an amino acid with a homologous amino acid residue, which is a residue that shares similar characteristics or properties. Homologous amino acids are known in the art, as are routine methods for making amino acid substitutions in antibody binding domains without significantly impacting binding or function of the antibody, see for instance, handbooks like Lehninger (Nelson, David L., and Michael M. Cox. 2017. Lehninger Principles of Biochemistry. 7th ed. New York, NY: W.H. Freeman) or Stryer (Berg, J., Tymoczko, J., Stryer, L. and Stryer, L., 2007. Biochemistry. New York: W.H. Freeman), incorporated herein in its entirety. In determining whether an amino acid can be replaced with a conserved amino acid, an assessment may typically be made of factors such as, but not limited to, (a) the structure of the polypeptide backbone in the area of the substitution, for example, a sheet or helical conformation, (b) the charge or hydrophobicity of the molecule at the target site, and/or (c) the bulk of the side chain(s). If a residue can be substituted with a residue which has common characteristics, such as a similar side chain or similar charge or hydrophobicity, then such a residue is preferred as a substitute. For example, the following groups can be determined: (1) non-polar: Ala (A), Gly (G), Val (V), Leu (L), Ile (I), Pro (P), Phe (F), Trp (W), Met (M); (2) uncharged polar: Ser (S), Thr (T), Cys (C), Tyr (Y), Asn (N), Gln (Q); (3) acidic: Asp (D), Glu (E); and (4) basic: Lys (K), Arg (R), His (H). Alternatively, the amino acids may be grouped as follows: (1) aromatic: Phe (F), Trp (W), Tyr (Y); (2) apolar: Leu (L), Val (V), Ile (I), Ala (A), Met (M); (3) aliphatic: Ala (A), Val (V), Leu (L), Ile (I); (4) acidic: Asp (D), Glu (E); (5) basic: His (H), Lys (K), Arg (R); and (6) polar: Gln (Q), Asn (N), Ser (S), Thr (T), Tyr (Y). Alternatively, amino acid residues may be divided into groups based on common side-chain properties: (1) hydrophobic: Met (M), Ala (A), Val (V), Leu (L), Ile (I); (2) neutral hydrophilic: Cys (C), Ser (S), Thr (T), Asn (N), Gln (Q); (3) acidic: Asp (D), Glu (E); (4) basic: His (H), Lys (K), Arg (R); (5) residues that influence chain orientation: Gly (G), Pro (P); and (6) aromatic: Trp (W), Tyr (Y), Phe (F).

The substitution of an amino acid residue with another present in the same group would be preferred. Accordingly, conservative amino acid substitution can involve exchanging a member of one of these classes for another member of that same class. Typically, the variation results in no, or substantially no, loss in binding specificity of the binding domain to its intended target.

Additional types of amino acid variations include variations resulting from somatic hypermutation or affinity maturation. Binding variants encompassed by the present disclosure include somatically hypermutated or affinity matured heavy chain variable regions, which are heavy chain variable regions derived from the same VH gene segments as the heavy chain variable regions described by sequence herein, the variants having amino acid variations, including non-conservative and/or conservative amino acid substitutions in one, two, or all three HCDRs. Routine methods for affinity maturing antibody binding domains are widely known in the art, see for instance Tabasinezhad M, et al. (Trends in therapeutic antibody affinity maturation: From in-vitro towards next-generation sequencing approaches. Immunol Lett. 2019 August; 212:106-113).

In certain embodiments, a polypeptide of the present disclosure comprises a heavy chain variable region having an amino acid sequence as set forth in SEQ ID NO: 1, or having at least 80%, or at least 85%, or at least 90%, or at least 95%, sequence identity thereto.

In certain embodiments, a polypeptide of the present disclosure also comprises variants, which, in addition to the variations in the HCDRs referred to above, comprise one or more variations in the framework regions. A variation can be any type of amino acid variation described herein, such as for instance a conservative amino acid substitution or non-conservative amino acid substitution resulting from somatic hypermutation or affinity maturation. In certain embodiments, a polypeptide of the present disclosure comprises no variations in the CDR regions but comprises one or more variations in the framework regions. Such variants have at least 80%, or at least 85%, or at least 90%, or at least 95% sequence identity to the sequences disclosed herein. Thus, in certain embodiments, a polypeptide of the present disclosure comprises:
a heavy chain variable region having at least 80%, or at least 85%, or at least 90%, or at least 95% sequence identity to the amino acid sequence as set forth in SEQ ID NO: 1, which heavy chain variable region comprises a HCDR1 amino acid sequence as set forth in SEQ ID NO: 2; a HCDR2 amino acid sequence as set forth in SEQ ID NO: 3; and a HCDR3 amino acid sequence as set forth in SEQ ID NO: 4.

In certain embodiments, the polypeptides of the present disclosure have been generated with the light chain VK1-39/JK1. A polypeptide of the present disclosure may be paired with any suitable light chain. In certain embodiments, a suitable light chain is light chain VK1-39/JK1. This light chain comprises a light chain variable region comprising light chain CDR1 (LCDR1), light chain CDR2 (LCDR2), and light chain CDR3 (LCDR3), having an amino acid sequence as set forth in SEQ ID NO: 6, SEQ ID NO: 7, and SEQ ID NO: 8, respectively. In certain embodiments, a suitable light chain comprises a light chain variable region having an amino acid sequence as set forth in SEQ ID NO: 5. In certain embodiments, a suitable light chain is the light chain of urelumab, cusatuzumab, fresolimumab, inotuzumab, lemzoparlimab, magrolimab, ofatumumab, olaratumab, omburtamab, gatipotuzumab (pankomab), tovetumab, trastuzumab, or nivolumab.

In certain embodiments, a polypeptide of the present disclosure further comprises a CH1 region. In certain embodiments, a polypeptide of the present disclosure further comprises a CH1 region, hinge, CH2 region, and CH3 region. A suitable CH1 region includes, but is not limited to, the CH1 region of which the amino acid sequence is set forth in SEQ ID NO: 33. A suitable hinge includes, but is not limited to, the hinge of which the amino acid sequence is set forth in SEQ ID NO: 32. Suitable CH2 and CH3 regions include, but are not limited to, the CH2 region of which the amino acid sequence is set forth in SEQ ID NO: 34 or 35, and the CH3 region of which the amino acid sequence is set forth in SEQ ID NO: 36.

A CH1, hinge, CH2, CH3, and/or CL may be modified according to methods known in the art in order to obtain favorable antibody characteristics, including for instance to promote heterodimerization of different heavy chains, to improve heavy-light chain pairing, and to enhance or reduce immune cell effector function. A CH3 region may comprise the terminal lysine residue, or lack the terminal lysine residue to improve manufacturability.

In certain embodiments, the present disclosure provides a CD3 binding domain comprising a polypeptide as described herein.

In certain embodiments, a CD3 binding domain of the present disclosure further comprises a polypeptide comprising a light chain variable region. In certain embodiments, said light chain variable region is an antibody light chain variable region.

In certain embodiments, a CD3 binding domain of the present disclosure is an antibody Fab domain.

In certain embodiments, the present disclosure provides a group of antigen-binding proteins that bind human CD3, wherein each antigen-binding protein within the group comprises a heavy chain variable region and light chain variable region, the heavy chain variable region comprising a HCDR3 having at least 70% sequence identity to the amino acid sequence as set forth in SEQ ID NO: 4. In certain embodiments, the present disclosure provides a group of antigen-binding proteins that bind human CD3, wherein each antigen-binding protein within the group comprises a heavy chain variable region and light chain variable region, the heavy chain variable region comprising a HCDR3 having at least 70% sequence identity to the amino acid sequence as set forth in SEQ ID NO: 4, and wherein the heavy chain variable region of each antigen-binding protein has the same HCDR3 length. In certain embodiments, the present disclosure provides a group of antigen-binding proteins that bind human CD3, wherein each antigen-binding protein within the group comprises a heavy chain variable region and light chain variable region, the heavy chain variable region comprising a HCDR3 having at least 70% sequence identity to the amino acid sequence as set forth in SEQ ID NO: 4, wherein the heavy chain variable region of each antigen-binding protein has the same HCDR3 length, and wherein the heavy chain variable region of each antigen-binding protein is derived from the same heavy chain variable region V gene segment.

In certain embodiments, the antigen-binding proteins are antibodies or antigen-binding fragments thereof.

The binding domains or antigen-binding proteins of the present disclosure can comprise any suitable light chain, including but not limited to common light chains known in the art. In certain embodiments, the binding domains or antigen-binding proteins of the present disclosure comprise common light chain VK1-39/JK1, or a variant thereof harboring a limited number, such as for instance one, two, or three non-conservative amino acid substitutions, or an unlimited number of conservative amino acid substitutions.

In certain embodiments, a binding domain or antigen-binding protein of the present disclosure comprises a light chain variable region having an amino acid sequence as set forth in SEQ ID NO: 5, or a variant thereof. In certain embodiments, a binding domain or antigen-binding protein of the present disclosure comprises a light chain variable region having an amino acid sequence as set forth in SEQ ID NO: 5, or a variant having at least 80%, or at least 85%, or at least 90%, or at least 95% sequence identity thereto.

In certain embodiments, a binding domain or antigen-binding protein of the present disclosure comprises a light chain variable region comprising light chain CDR1 (LCDR1), light chain CDR2 (LCDR2), and light chain CDR3 (LCDR3), having an amino acid sequence as set forth in SEQ ID NO: 6, SEQ ID NO: 7, and SEQ ID NO: 8, respectively. In certain embodiments, the light chain variable region of a binding domain or antigen-binding protein of the present disclosure also includes variants thereof, wherein each of the LCDRs may comprise one, two, or three amino acid variations. In certain embodiments, the amino acid variation is a conservative amino acid substitution.

A light chain or light chain variable region comprising these LCDRs and/or light chain variable region can be, for example, the light chain referred to in the art as VK1-39/JK1. This is a common light chain. The term ‘common light chain’ according to the present disclosure refers to a light chain that is capable of pairing with multiple different heavy chains, such as for instance heavy chains having different antigen or epitope binding specificities. A common light chain is particularly useful in the generation of, for instance, bispecific or multispecific antibodies, where antibody production is more efficient when all binding domains comprise the same light chain. The term “common light chain” encompasses light chains that are identical or have some amino acid sequence differences while the binding specificity of the full length antibody is not affected. It is for instance possible within the scope of the definition of common light chains as used herein, to prepare or find light chains that are not identical but still functionally equivalent, e.g., by using well established variations that introduce conservative amino acid changes, changes of amino acids in regions that are known to or are shown to not or only partly contribute to binding specificity when paired with the heavy chain, and the like.

Apart from a common light chain comprising the LCDRs and/or light chain variable region referred to above, other common light chains known in the art may be used. Examples of such common light chains include, but are not limited to: VK1-39/JK5, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 13. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 13, wherein each of the LCDRs may comprise one, two, or three amino acid variations, for example substitutions. In certain embodiments, the light chain comprises a light chain variable region having an amino acid sequence as set forth in SEQ ID NO: 13, or having at least 80%, or at least 85%, or at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 14, SEQ ID NO: 15, and SEQ ID NO: 16, respectively; VK3-15/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 18. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 18, wherein each of the LCDRs may comprise one, two, or three amino acid variations, for example substitutions. In certain embodiments, the light chain comprises a light chain variable region having an amino acid sequence as set forth in SEQ ID NO: 18, or having at least 80%, or at least 85%, or at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 19, SEQ ID NO: 20, and SEQ ID NO: 21, respectively; VK3-20/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 23. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 23, wherein each of the LCDRs may comprise one, two, or three amino acid variations, for example substitutions. In certain embodiments, the light chain comprises a light chain variable region having an amino acid sequence as set forth in SEQ ID NO: 23, or having at least 80%, or at least 85%, or at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 24, SEQ ID NO: 25, and SEQ ID NO: 26, respectively; and VL3-21/JL3, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 28. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 28, wherein each of the LCDRs may comprise one, two, or three amino acid variations, for example substitutions. In certain embodiments, the light chain comprises a light chain variable region having an amino acid sequence as set forth in SEQ ID NO: 28, or having at least 80%, or at least 85%, or at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 29, SEQ ID NO: 30, and SEQ ID NO: 31, respectively.

VK1-39 is short for Immunoglobulin Variable Kappa 1-39 Gene. The gene is also known as Immunoglobulin Kappa Variable 1-39; IGKV139; IGKV1-39; IgVκ1-39. External Ids for the gene are HGNC: 5740; Entrez Gene: 28930; Ensembl: ENSG00000242371. An amino acid sequence for VK1-39 is given as SEQ ID NO: 11. This is the sequence of the V-region. The V-region can be combined with one of five J-regions. Suitable VJ-region sequences are indicated as VK1-39/JK1 (SEQ ID NO: 12) and VK1-39/JK5 (SEQ ID NO: 13); alternative names are IgVκ1-39*01/IGJκ1*01 or IgVκ1-39*01/IGJκ5*01 (nomenclature according to the IMGT database worldwide web at imgt.org). These names are exemplary and encompass allelic variants of the gene segments.

VK3-15 is short for Immunoglobulin Variable Kappa 3-15 Gene. The gene is also known as Immunoglobulin Kappa Variable 3-15; IGKV315; IGKV3-15; IgVκ3-15. External Ids for the gene are HGNC: 5816; Entrez Gene: 28913; Ensembl: ENSG00000244437. An amino acid sequence for VK3-15 is given as SEQ ID NO: 17. This is the sequence of the V-region. The V-region can be combined with one of five J-regions. A suitable VJ-region sequence is indicated as VK3-15/JK1 (SEQ ID NO: 18); alternative name is Vκ3-15*01/IGJκ1*01 (nomenclature according to the IMGT database worldwide web at imgt.org). This name is exemplary and encompasses allelic variants of the gene segments.

VK3-20 is short for Immunoglobulin Variable Kappa 3-20 Gene. The gene is also known as Immunoglobulin Kappa Variable 3-20; IGKV320; IGKV3-20; IgVκ3-20. External Ids for the gene are HGNC: 5817; Entrez Gene: 28912; Ensembl: ENSG00000239951. An amino acid sequence for VK3-20 is indicated as SEQ ID NO: 22. This is the sequence of the V-region. The V-region can be combined with one of five J-regions. A suitable VJ-region sequence is indicated as VK3-20/JK1 (SEQ ID NO: 23); alternative name is IgVκ3-20*01/IGJκ1*01 (nomenclature according to the IMGT database worldwide web at imgt.org). This name is exemplary and encompasses allelic variants of the gene segments.

VL3-21 is short for Immunoglobulin Variable Lambda 3-21 Gene. The gene is also known as Immunoglobulin Lambda Variable 3-21; IGLV321; IGLV3-21; IgVλ3-21. External Ids for the gene are HGNC: 5905; Entrez Gene: 28796; Ensembl: ENSG00000211662.2. An amino acid sequence for VL3-21 is given as SEQ ID NO: 27. This is the sequence of the V-region. The V-region can be combined with one of five J-regions. A suitable VJ-region sequence is indicated as VL3-21/JL3 (SEQ ID NO: 28); alternative name is IgVλ3-21/IGJλ3 (nomenclature according to the IMGT database worldwide web at imgt.org). This name is exemplary and encompasses allelic variants of the gene segments.

Further, any light chain variable region of an antibody available in the art may be used, as may any other light chain variable region that can readily be obtained, such as from, for instance, an antibody display library by showing antigen binding activity when paired with a polypeptide of the present disclosure.

In certain embodiments, the binding domains or antigen-binding proteins of the present disclosure comprise the light chain, or light chain variable region, of urelumab, cusatuzumab, fresolimumab, inotuzumab, lemzoparlimab, magrolimab, ofatumumab, olaratumab, omburtamab, gatipotuzumab (pankomab), tovetumab, trastuzumab, or nivolumab. The sequences of the variable regions of these light chains are provided herein. The light chain variable regions as used in accordance with the present disclosure may comprise one or more sequence variations that do not significantly alter the binding and functional activity of the resulting antibody, i.e. equivalent light chain variable regions.

In certain embodiments, a CD3 binding domain of the present disclosure or an antigen-binding protein within a group of antigen-binding proteins of the present disclosure comprises a light chain of which the light chain variable region comprises the CDR1, CDR2, and CDR3 sequences of a light chain selected from the light chains of urelumab, cusatuzumab, fresolimumab, inotuzumab, lemzoparlimab, magrolimab, ofatumumab, olaratumab, omburtamab, gatipotuzumab (pankomab), tovetumab, trastuzumab and nivolumab. The CDR sequences can be determined in accordance with any numbering system known in the art, including, but not limited to, IMGT and Kabat. The CDR sequences according to IMGT are indicated in the list of sequences herein in italic; the CDR sequences according to Kabat are underlined.

In certain embodiments, a CD3 binding domain of the present disclosure or an antigen-binding protein within a group of antigen-binding proteins of the present disclosure comprises a light chain of which the light chain variable region comprises:
the light chain CDR1 (LCDR1), light chain CDR2 (LCDR2), and light chain CDR3 (LCDR3), of a light chain variable region having an amino acid sequence as set forth in SEQ ID NO: 5;
the light chain CDR1 (LCDR1), light chain CDR2 (LCDR2), and light chain CDR3 (LCDR3), of a light chain variable region having an amino acid sequence as set forth in SEQ ID NO: 41;
the light chain CDR1 (LCDR1), light chain CDR2 (LCDR2), and light chain CDR3 (LCDR3), of a light chain variable region having an amino acid sequence as set forth in SEQ ID NO: 42;
the light chain CDR1 (LCDR1), light chain CDR2 (LCDR2), and light chain CDR3 (LCDR3), of a light chain variable region having an amino acid sequence as set forth in SEQ ID NO: 43 or SEQ ID NO: 68;
the light chain CDR1 (LCDR1), light chain CDR2 (LCDR2), and light chain CDR3 (LCDR3), of a light chain variable region having an amino acid sequence as set forth in SEQ ID NO: 45 or SEQ ID NO: 69;
the light chain CDR1 (LCDR1), light chain CDR2 (LCDR2), and light chain CDR3 (LCDR3), of a light chain variable region having an amino acid sequence as set forth in SEQ ID NO: 49;
the light chain CDR1 (LCDR1), light chain CDR2 (LCDR2), and light chain CDR3 (LCDR3), of a light chain variable region having an amino acid sequence as set forth in SEQ ID NO: 50;
the light chain CDR1 (LCDR1), light chain CDR2 (LCDR2), and light chain CDR3 (LCDR3), of a light chain variable region having an amino acid sequence as set forth in SEQ ID NO: 52;
the light chain CDR1 (LCDR1), light chain CDR2 (LCDR2), and light chain CDR3 (LCDR3), of a light chain variable region having an amino acid sequence as set forth in SEQ ID NO: 53;
the light chain CDR1 (LCDR1), light chain CDR2 (LCDR2), and light chain CDR3 (LCDR3), of a light chain variable region having an amino acid sequence as set forth in SEQ ID NO: 56;
the light chain CDR1 (LCDR1), light chain CDR2 (LCDR2), and light chain CDR3 (LCDR3), of a light chain variable region having an amino acid sequence as set forth in SEQ ID NO: 57;
the light chain CDR1 (LCDR1), light chain CDR2 (LCDR2), and light chain CDR3 (LCDR3), of a light chain variable region having an amino acid sequence as set forth in SEQ ID NO: 58 or SEQ ID NO: 70;
the light chain CDR1 (LCDR1), light chain CDR2 (LCDR2), and light chain CDR3 (LCDR3), of a light chain variable region having an amino acid sequence as set forth in SEQ ID NO: 59 or SEQ ID NO: 71; or
the light chain CDR1 (LCDR1), light chain CDR2 (LCDR2), and light chain CDR3 (LCDR3), of a light chain variable region having an amino acid sequence as set forth in SEQ ID NO: 64 or SEQ ID NO: 72.

In certain embodiments, a CD3 binding domain of the present disclosure or an antigen-binding protein within a group of antigen-binding proteins of the present disclosure comprises a light chain of which the light chain variable region comprises:
the light chain CDR1 (LCDR1) of SEQ ID NO: 6, the light chain CDR2 (LCDR2) of SEQ ID NO: 7, and the light chain CDR3 (LCDR3) of SEQ ID NO: 8;
the light chain CDR1 (LCDR1) of SEQ ID NO: 73, the light chain CDR2 (LCDR2) of SEQ ID NO: 74, and the light chain CDR3 (LCDR3) of SEQ ID NO: 75;
the light chain CDR1 (LCDR1) of SEQ ID NO: 76, the light chain CDR2 (LCDR2) of SEQ ID NO: 77, and the light chain CDR3 (LCDR3) of SEQ ID NO: 78;
the light chain CDR1 (LCDR1) of SEQ ID NO: 79, the light chain CDR2 (LCDR2) of SEQ ID NO: 80, and the light chain CDR3 (LCDR3) of SEQ ID NO: 81 or SEQ ID NO: 82;
the light chain CDR1 (LCDR1) of SEQ ID NO: 83, the light chain CDR2 (LCDR2) of SEQ ID NO: 84, and the light chain CDR3 (LCDR3) of SEQ ID NO: 85;
the light chain CDR1 (LCDR1) of SEQ ID NO: 86, the light chain CDR2 (LCDR2) of SEQ ID NO: 87, and the light chain CDR3 (LCDR3) of SEQ ID NO: 88;
the light chain CDR1 (LCDR1) of SEQ ID NO: 89, the light chain CDR2 (LCDR2) of SEQ ID NO: 90, and the light chain CDR3 (LCDR3) of SEQ ID NO: 91;
the light chain CDR1 (LCDR1) of SEQ ID NO: 92, the light chain CDR2 (LCDR2) of SEQ ID NO: 93, and the light chain CDR3 (LCDR3) of SEQ ID NO: 94;
the light chain CDR1 (LCDR1) of SEQ ID NO: 95, the light chain CDR2 (LCDR2) of SEQ ID NO: 96, and the light chain CDR3 (LCDR3) of SEQ ID NO: 97;
the light chain CDR1 (LCDR1) of SEQ ID NO: 98, the light chain CDR2 (LCDR2) of SEQ ID NO: 99, and the light chain CDR3 (LCDR3) of SEQ ID NO: 100;
the light chain CDR1 (LCDR1) of SEQ ID NO: 101, the light chain CDR2 (LCDR2) of SEQ ID NO: 102, and the light chain CDR3 (LCDR3) of SEQ ID NO: 103;
the light chain CDR1 (LCDR1) of SEQ ID NO: 104, the light chain CDR2 (LCDR2) of SEQ ID NO: 105, and the light chain CDR3 (LCDR3) of SEQ ID NO: 106;
the light chain CDR1 (LCDR1) of SEQ ID NO: 107, the light chain CDR2 (LCDR2) of SEQ ID NO: 108, and the light chain CDR3 (LCDR3) of SEQ ID NO: 109; or
the light chain CDR1 (LCDR1) of SEQ ID NO: 110, the light chain CDR2 (LCDR2) of SEQ ID NO: 111, and the light chain CDR3 (LCDR3) of SEQ ID NO: 112.

In certain embodiments, the light chain variable region of a CD3 binding domain or an antigen-binding protein of the present disclosure also includes variants thereof, wherein each of the LCDRs may comprise one, two, or three amino acid variations. In certain embodiments, the light chain variable region of a CD3 binding domain or an antigen-binding protein of the present disclosure also includes variants thereof, wherein each of the LCDRs may contain one, two, or at most three amino acid variations. In certain embodiments, the amino acid variation is a conservative amino acid substitution.

In certain embodiments, the light chain variable region of a CD3 binding domain or antigen-binding protein of the present disclosure comprises an amino acid sequence as set forth in SEQ ID NO: 5; 41; 42; 43; 45; 49; 50; 52; 53; 56; 57; 58; 59; or 64, or having at least 80%, or at least 85%, or at least 90%, or at least 95%, sequence identity thereto. In certain embodiments, a binding domain or antigen-binding protein of the present disclosure also includes variants, which, in addition to the variations in the LCDRs referred to above, comprise one or more variations in the framework regions. A variation is preferably a conservative amino acid substitution. In certain embodiments, a binding domain or antigen-binding protein of the present disclosure comprises no variations in the LCDR regions but comprises one or more variations in the framework regions. Such variants have at least 80%, or at least 85%, or at least 90%, or at least 95% sequence identity to the sequences disclosed herein.

In certain embodiments, a CD3 binding domain or an antigen-binding protein within a group of antigen-binding proteins of the present disclosure may further comprise a CL region. Any CL domain may be used, in particular a human CL. An example of a suitable CL domain is provided by the amino acid sequence provided as SEQ ID NO: 9.

In certain embodiments, the present disclosure provides a binding moiety comprising a polypeptide as described herein, or a CD3 binding domain as described herein.

A “binding moiety” refers to a proteinaceous molecule and includes for instance all antibody formats available in the art, such as for example a full length IgG antibody, immunoconjugates, diabodies, BiTEs, Fab fragments, scFv, tandem scFv, single domain antibody (like VHH and VH), minibodies, scFab, scFv-zipper, nanobodies, DART molecules, TandAb, Fab-scFv, F(ab)′2, F(ab)′2-scFv2, and intrabodies, as well as any other antibody formats known to a person of ordinary skill in the art.

In certain embodiments, a binding moiety of the present disclosure is a monospecific binding moiety, in particular a monospecific antibody. A monospecific antibody according to the present disclosure is an antibody, in any antibody format, that comprises one or more binding domains with specificity for a single target. In certain embodiments, a monospecific binding moiety of the present disclosure is a bivalent monospecific antibody. In certain embodiments, a monospecific binding moiety of the present disclosure may further comprise an Fc region or a part thereof. In certain embodiments, a monospecific binding moiety of the present disclosure is an IgG1 antibody.

In certain embodiments, a binding moiety of the present disclosure is a multispecific antibody. A multispecific antibody according to the present disclosure is an antibody that comprises at least two binding domains which have specificity for at least two different targets or epitopes. In certain embodiments, a multispecific antibody of the present disclosure is a bispecific antibody. In certain embodiments, a multispecific antibody of the present disclosure is a bivalent bispecific antibody. In certain embodiments, a multispecific antibody of the present disclosure is a trivalent bispecific antibody. In certain embodiments, a multispecific antibody of the present disclosure is a trispecific antibody. In certain embodiments, a multispecific antibody of the present disclosure is a trivalent trispecific antibody. In certain embodiments, a multispecific antibody of the present disclosure may further comprise an Fc region or a part thereof. In certain embodiments, a multispecific binding moiety of the present disclosure is an IgG1 antibody.

An “Fc region” typically comprises a hinge, CH2, and CH3 region. Suitable hinge, CH2, and CH3 regions are as described herein. The Fc region mediates effector functions of an antibody, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function.

In certain embodiments, a binding moiety comprising a polypeptide or binding domain of the present disclosure has Fc effector function. In certain embodiments, a binding moiety comprising a polypeptide or binding domain of the present disclosure has enhanced Fc effector function. In certain embodiments, a binding moiety comprising a polypeptide or binding domain of the present disclosure exhibits antibody-dependent cell-mediated cytotoxicity (ADCC).

A binding moiety, such as an antibody, can be engineered to enhance the ADCC activity (for review, see Kubota T et al. Cancer Sci. 2009; 100(9):1566-72). For instance, ADCC activity of an antibody can be improved when the antibody itself has a low ADCC activity, by slightly modifying the constant region of the antibody (Junttila T T. et al. Cancer Res. 2010; 70(11):4481-9). Changes are sometimes also made to improve storage or production or to remove C-terminal lysines (Kubota T et al. Cancer Sci. 2009; 100(9):1566-72). Another way to improve ADCC activity of an antibody is by enzymatically interfering with the glycosylation pathway resulting in a reduced fucose (von Horsten H H. et al. Glycobiology. 2010; 20(12): 1607-18). Alternatively, or additionally, multiple other strategies can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. In certain embodiments, a binding moiety of the present disclosure exhibits enhanced antibody-dependent cell-mediated cytotoxicity (ADCC). In certain embodiments, a binding moiety of the present disclosure is afucosylated.

Constant regions of a binding moiety of the present disclosure may comprise one or more variations that modulate properties of the binding moiety other than its binding properties to the target antigens. For instance, the constant regions of a multispecific binding moiety may comprise one or more variations that favor heterodimerization of two different heavy chains over homodimerization, and/or the constant regions of a binding moiety may comprise one or more variations that reduce or improve effector function, preferably one or more variations that reduce effector function.

In certain embodiments, the present disclosure provides a nucleic acid useful for producing a polypeptide, binding domain, or binding moiety, of the present disclosure. In certain embodiments, such nucleic acid comprises a nucleic acid sequence encoding a polypeptide as described herein. In certain embodiments, a nucleic acid of the present disclosure may further comprise a nucleic acid sequence encoding a CH1 region and preferably a hinge, CH2 and CH3 region. In certain embodiments, a nucleic acid of the present disclosure may further comprise at least one nucleic acid sequence encoding a light chain variable region, and preferably a CL region. In certain embodiments, the light chain variable region can be a light chain variable region as described herein.

In certain embodiments, the present disclosure provides a vector comprising a nucleic acid of the present disclosure useful for producing a binding domain or binding moiety of the present disclosure. In certain embodiments, such vector comprises a nucleic acid sequence encoding a polypeptide as described herein. In certain embodiments, a vector of the present disclosure may further comprise a nucleic acid sequence encoding a CH1 region and preferably a hinge, CH2 and CH3 region. In certain embodiments, a vector of the present disclosure may further comprise at least one nucleic acid sequence encoding a light chain variable region, and preferably a CL region. In certain embodiments, the light chain variable region can be a light chain variable region as described herein.

In certain embodiments, the present disclosure also provides a cell comprising a vector as described herein. In certain embodiments, the present disclosure also provides a cell comprising a nucleic acid, for example a vector, comprising a sequence that encodes a polypeptide as described herein. In certain embodiments, such nucleic acid, for example a vector, may further comprise a nucleic acid sequence that encodes a CH1 region and preferably a hinge, CH2 and CH3 region. In certain embodiments, such nucleic acid, for example a vector, may further comprise a nucleic acid sequence that encodes a light chain variable region, and preferably a CL region. In certain embodiments, the light chain variable region can be a light chain variable region as described herein.

In certain embodiments, the present disclosure also provides a cell producing a polypeptide, binding domain, or binding moiety as described herein. In certain embodiments, such cell can be a recombinant cell, which has been transformed with nucleic acid, for example a vector, of the present disclosure. In certain embodiments, a cell of the present disclosure comprises a nucleic acid sequence, for example a vector, comprising a sequence that encodes a polypeptide as described herein. In certain embodiments, said nucleic acid sequence, for example a vector, further comprises a nucleic acid sequence that encodes a CH1 region and preferably a hinge, CH2 and CH3 region. In certain embodiments, a cell of the present disclosure further comprises at least one nucleic acid, for example a vector, comprising a sequence that encodes a light chain variable region, in particular a light chain variable region as described herein, and preferably a CL region.

In certain embodiments, the present disclosure provides a pharmaceutical composition comprising an effective amount of a polypeptide as described herein, or a CD3 binding domain as described herein, or a binding moiety as described herein, and a pharmaceutically acceptable carrier.

In certain embodiments, the present disclosure provides a polypeptide as described herein, or a CD3 binding domain as described herein, or a binding moiety as described herein, or a pharmaceutical composition as described herein, for use in therapy.

In certain embodiments, the present disclosure provides a polypeptide as described herein, or a CD3 binding domain as described herein, or a binding moiety as described herein, or a pharmaceutical composition as described herein, for use in the treatment of cancer.

In certain embodiments, the present disclosure provides a method for treating a disease, comprising administering an effective amount of a polypeptide as described herein, or a CD3 binding domain as described herein, or a binding moiety as described herein, or a pharmaceutical composition as described herein, to an individual in need thereof.

In certain embodiments, the present disclosure provides a method for treating cancer, comprising administering an effective amount of a polypeptide as described herein, or a CD3 binding domain as described herein, or a binding moiety as described herein, or a pharmaceutical composition as described herein, to an individual in need thereof.

As used herein, the terms “individual”, “subject” and “patient” are used interchangeably and refer to a mammal such as a human, mouse, rat, hamster, guinea pig, rabbit, cat, dog, monkey, cow, horse, pig and the like, and in particular to a human subject having cancer.

The terms “treat,” “treating,” and “treatment,” as used herein, refer to any type of intervention or process performed on or administering an active agent or combination of active agents to a subject with the objective of curing or improving a disease or symptom thereof or which produces a positive therapeutic response. As used herein, “positive therapeutic response” refers to a treatment producing a beneficial effect, e.g. reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease, such as, for example, amelioration of at least one symptom of a disease or disorder, e.g. cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease tumor size, decrease the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that treats a disease, such as cancer. In some embodiments, a therapeutic amount is an amount sufficient to delay tumor development. In some embodiments, a therapeutic amount is an amount sufficient to prevent or delay tumor recurrence.

As used herein, an effective amount of the agent or composition is one that, for example, may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

An effective amount may vary according to factors such as the disease state, age, sex, and weight of the individual to be treated, and the ability of the agent or combination of agents to elicit a desired response in the individual, which can be readily evaluated by the ordinarily skilled physician or other health care worker.

An effective amount can be administered to a subject in one or more administrations.

An effective amount can also include an amount that balances any toxic or detrimental effects of the agent or combination of agents and the beneficial effects.

The term “agent” refers to a therapeutically active substance, in the present case a polypeptide, binding domain, or binding moiety of the present disclosure, or a pharmaceutical composition of the present disclosure.

As used herein, “to comprise” and its conjugations is used in its non-limiting sense to mean that items following the word are included, but items not specifically mentioned are not excluded.

The articles “a” and “an” are used herein to refer to one or more of the grammatical object of the article. By way of example, “an element” means one or more elements.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

Note that in the present specification, unless stated otherwise, amino acid positions assigned to HCDRs and frameworks in a variable region of an antibody or antibody fragment are specified according to Kabat's numbering (see Sequences of Proteins of Immunological Interest (National Institute of Health, Bethesda, Md., 1987 and 1991)) and the numbering for LCDRs and frameworks in a variable region of an antibody or antibody fragment are according to Kabat or IMGT (discussed in Giudicelli et al., Nucleic Acids Res. 25: 206-21 1 1997). Amino acids in the constant regions are indicated according to the EU numbering system.

Accession numbers are primarily given to provide a further method of identification of a target, the actual sequence of the protein bound may vary, for instance because of a mutation in the encoding gene such as those occurring in some cancers or the like. An antigen binding site of a binding domain or binding moiety of the disclosure can bind the antigen and a variety of variants thereof, such as those expressed by some antigen positive immune or tumor cells. HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensembl provides the accession number with which information on the gene or protein encoded by the gene can be obtained from the Ensembl database. Ensembl is a joint project between EMBL-EBI and the Wellcome Trust Sanger Institute to develop a software system which produces and maintains automatic annotation on selected eukaryotic genomes.

When herein reference is made to a gene or a protein, the reference is preferably to the human form of the gene or protein. When herein reference is made to a gene or protein reference is made both to the natural gene or protein and to variant forms of the gene or protein as can be detected in tumors, cancers and the like, preferably as can be detected in human tumors, cancers and the like.

### BRIEF DESCRIPTION OF THE DRAWINGS

FIG. 1—Binding of SC5Ab1 combined with the cLC or the light chain of olaratumab, ofatumumab, urelumab, cusatuzumab, inotuzumab, fresolimumab, trastuzumab, or daratumumab on HPB-ALL cells, measured as mean fluorescence intensity (MFI) by FACS at different IgG concentrations.

FIG. 2—A) Digital capillary electrophoresis gel image of SC5Ab1 combined with the cLC or the light chain of cusatuzumab, fresolimumab, ofatumumab, or trastuzumab; B) Electropherogram of SC5Ab1 combined with the light chain of fresolimumab.

FIG. 3—Binding of SC5Ab1 combined with the cLC or the light chain of cusatuzumab, fresolimumab, ofatumumab, or trastuzumab on HPB-ALL cells, measured as mean fluorescence intensity (MFI) by FACS at different IgG concentrations.

FIG. 4—Thermostability of IgGs comprising the VH region of antibody SC5Ab1 paired with the VL region of the cLC or with the light chain of cusatuzumab, fresolimumab, ofatumumab, or trastuzumab. Stability was characterized by Tm (continuous lines) and Tagg (dotted lines). Tm was measured by the change of intrinsic protein fluorescence, displayed as Barycentric mean (BCM) at a spectral range between 250 and 720 nm and the aggregation temperature Tagg was measured by static light scattering (SLS) at 266 nm using UNcle system (Unchained Labs).

### EXAMPLES

In the Examples, which are used to illustrate the present disclosure but are not intended to limit the disclosure in any way, the binding domains are screened in IgG1 format, comprising a heavy chain comprising a heavy chain variable region having an amino acid sequence as set forth in SEQ ID NO: 1, a CH1 having an amino acid sequence as set forth in SEQ ID NO: 33, a hinge having an amino acid sequence as set forth in SEQ ID NO: 32, a CH2 having an amino acid sequence as set forth in SEQ ID NO: 34, and a CH3 having an amino acid sequence as set forth in SEQ ID NO: 36, and a light chain comprising a light chain variable region as further specified herein and a CL having an amino acid sequence as set forth in SEQ ID NO: 9.

Control antibodies used in the Examples include:
Positive control anti-CD3 antibody, which is a bivalent monospecific antibody comprising two heavy chains having an amino acid sequence as set forth in SEQ ID NO: 37 and two light chains having an amino acid sequence as set forth in SEQ ID NO: 66;
Negative control IgG1 antibody (RSV-G), which is a bivalent monospecific antibody comprising two heavy chains having an amino acid sequence as set forth in SEQ ID NO: 38 and two light chains having an amino acid sequence as set forth in SEQ ID NO: 10;
Negative control IgG1 antibody (TT), which is a bivalent monospecific antibody comprising two heavy chains having an amino acid sequence as set forth in SEQ ID NO: 39 and two light chains having an amino acid sequence as set forth in SEQ ID NO: 10.

### Example 1—Pairing of CD3 Heavy Chains with the Light Chains of Cetuximab, Trastuzumab, and Nivolumab

The aim of this study was to assess if the heavy chains of CD3 antibodies generated with a light chain having an amino acid as set forth in SEQ ID NO: 10 can be combined with other light chains while conserving the specificity and affinity of the original antibodies.

Binding domains, antibodies and heavy chain variable regions with binding specificity to human CD3 were obtained by immunizing transgenic mice comprising a common IGKV1-39 light chain (MeMo® mice) with human CD3 antigenic moieties, including the use of TCR/CD3 containing lipoparticles (for example as described in WO 2020/204708), different forms of DNA, protein and cell-based antigen delivery. Antibodies that bind human CD3 were grouped in different superclusters based on the same VH V-gene segment usage and having at least 70% sequence identity in the HCDR3 and the same HCDR3 length.

The heavy chain variable region (VH) sequence of one representant of each supercluster was combined with the light chain variable region (VL) sequence of cetuximab, trastuzumab, and nivolumab. These three commercially available antibodies bind different targets and have diverse VLs, as shown in Table 1. The VH sequences were also combined with the light chain variable region having an amino acid sequence as set forth in SEQ ID NO: 5, to serve as parental control.

TABLE 1
Commercial antibodies selected for combination with CD3 VHs.

| Light chain | Target | VL | VL sequence |
|---|---|---|---|
| Cetuximab | EGFR | IGKV5-48*01_mo | SEQ ID NO: 40 |
| Trastuzumab | HER2 | IGKV1-39*01_hu | SEQ ID NO: 41 |
| Nivolumab | PD-1 | IGKV3-11*01_hu | SEQ ID NO: 42 |
| Parental | — | IGKV1-39*01_hu | SEQ ID NO: 5 |

Abbreviations: mo—mouse, hu—human.

### ELISA

Binding of the CD3 antibodies to CD3-containing virus-like particles (VLPs) was assessed by ELISA.

HEK293 huCD3 (Integral Molecular; Cat. No. INT-2131B), HEK293 cyCD3 (Integral Molecular; Cat. No. INT-2132B), and HEK293 “null” (Integral Molecular; Cat. No. INT-2128B) biotinylated VLPs were coated overnight on ELISA plates at 4° C. at 5 units (U)/well. Wells were blocked with 4% skimmed milk (Marvel) in 1×PBS. CD3 antibodies or control antibodies in 1% skimmed milk/1×PBS were added to the huCD3 and cyCD3 bio-VLPs using seven step, three-fold dilutions starting at 5 g/ml. CD3 antibodies or control antibodies in 1% skimmed milk/1×PBS were added to the “null” bio-VLPs at a single concentration of 5 μg/ml. Antibodies were incubated for 1 hour at room temperature (RT).

Control antibodies include: positive control anti-CD3 antibody used to confirm coating of the CD3-containing bio-VLPs, and a negative control IgG1 (RSV-G) antibody used to confirm specificity of binding. Control antibodies were used at 5 μg/ml.

Antibodies were detected by a goat anti-huIgG (Fc) HRP-conjugated secondary antibody (Bethyl Labs; Cat. No. A80-104P) in 1% skimmed milk/1×PBS, at 1:2000, incubated for 1 hour at RT.

Wells were washed three times with 1×PBS in between steps and 5 times with 1×PBS prior to development. All washing steps were performed using buffers without Tween-20. Development involved the addition of 100 μl of TMB solution (eBioscience, Cat. No. 00-4201-56). Reaction was stopped with 100 μl of 0.5M (1N) H₂SO₄ (Fisher Chemical, Cat. No. J/8430/15).

Read-out in O.D. 450 nm using BioTek Elx808 ELISA plate reader. ELISA titration data was analyzed in GraphPad Prism.

Antibodies that showed binding in ELISA, as compared to its respective parental control, were further screened in FACS.

### FACS

Binding and relative affinity of the CD3 antibodies to huCD3 was determined by FACS.

293FF cells transiently transfected to express (hu)CD3: TCR cultured in FreeStyle™ 293 Expression Medium (Gibco, Cat. No. 12338-018) were seeded at 0.5×10⁶ cells/well; and HPB-ALL cells endogenously expressing huCD3 cultured in RPMI 1640 medium (Gibco, Cat. No. 21875-091) with FBS (Gibco, Cat. No. A3160801) and penstrep (Gibco, Cat. No. 15140-122) were seeded at 1×10⁶ cells/well.

CD3 antibodies or positive control anti-CD3 antibody were added using eight step, semi-log (3.16 fold) serial dilutions starting at 10 μg/ml. Negative control IgG1 antibody (RSV-G) was added at a single concentration of 10 μg/ml. Antibodies were incubated for 30 minutes on ice in FACS buffer (0.5% FBS/EDTA 1:1000/1×PBS).

Antibodies were detected by a goat anti-huIgG PE secondary antibody (Invitrogen, Cat. No. H10104), at 1:100, incubated for 30 minutes on ice in FACS buffer (0.5% FBS/EDTA 1:1000/1×PBS).

FACS analysis was performed (BD Accuri™) and the mean fluorescence intensity (MFI) of each antibody was plotted as a function of the log of antibody concentration using GraphPad prism software using non-linear regression, asymmetric (five parameters) equation with robust fit.

AUC values were determined and used to assess the relative affinity to huCD3 of the antibodies. FACS results were considered positive when MFI values were more than two-fold higher than the negative control IgG1 antibody (RSV-G).

An overview of the results of the ELISA and FACS are shown in Table 2. A select number of antibodies retain CD3 binding when combined with one or more of the light chains of cetuximab, trastuzumab, and nivolumab, albeit with a lower affinity than when the VHs are paired with their original light chain (cLC). Antibody SC5Ab1, which comprises a heavy chain variable region having an amino acid sequence as set forth in SEQ ID NO: 1, when combined with the light chain variable region of nivolumab shows a similar or higher binding to CD3 than when combined with its original light chain.

TABLE 2
Results of the ELISA and FACS.

| | | | FACS | |
|---|---|---|---|---|
| | | ELISA HEK293 | huCD3:TCR | |
| | | huCD3 Bio-VLPs | 293FF | FACS HBP-ALL |
| | | AUC compared | AUC compared | AUC compared |
| CD3 antibody | VL | to cLC (%) | to cLC (%) | to cLC (%) |
| SC1Ab1 | Cetuximab | N.D. | 10.38 | N.D. |
| SC2Ab1 | (SEQ ID | 34.02 | N.D. | 5.76 |
| SC3Ab1 | NO: 40) | 47.11 | 29.72 | 17.73 |
| SC4Ab1 | | N.D. | N.D. | N.D. |
| SC5Ab1 | | 13.39 | N.D. | N.D. |
| SC6Ab1 | | N.D. | N.D. | N.D. |
| SC7Ab1 | | N.D. | N.D. | N.D. |
| SC8Ab1 | | N.D. | N.D. | N.D. |
| SC1Ab1 | Trastuzumab | N.D. | N.D. | N.D. |
| SC2Ab1 | (SEQ ID | 23.79 | 90.12 | N.D. |
| SC3Ab1 | NO: 41) | 64.02 | 55.54 | 8.69 |
| SC4Ab1 | | 9.01 | N.D. | N.D. |
| SC5Ab1 | | 50.98 | 28.65 | 7.21 |
| SC6Ab1 | | 10.52 | N.D. | N.D. |
| SC7Ab1 | | N.D. | N.D. | N.D. |
| SC8Ab1 | | N.D. | N.D. | N.D. |
| SC1Ab1 | Nivolumab | N.D. | N.D. | N.D. |
| SC2Ab1 | (SEQ ID | N.D. | N.D. | N.D. |
| SC3Ab1 | NO: 42) | N.D. | N.D. | N.D. |
| SC4Ab1 | | 6.12 | N.D. | N.D. |
| SC5Ab1 | | 97.14 | 169.41 | 96.94 |
| SC6Ab1 | | N.D. | N.D. | N.D. |
| SC7Ab1 | | 42.92 | N.D. | N.D. |
| SC8Ab1 | | N.D. | N.D. | N.D. |
| SC1Ab1 | cLC | 100 | 100 | 100 |
| SC2Ab1 | (SEQ ID | 100 | 100 | 100 |
| SC3Ab1 | NO: 5) | 100 | 100 | 100 |
| SC4Ab1 | | 100 | 100 | 100 |
| SC5Ab1 | | 100 | 100 | 100 |
| SC6Ab1 | | 100 | 100 | 100 |
| SC7Ab1 | | 100 | 100 | 100 |
| SC8Ab1 | | 100 | 100 | 100 |

N.D. Not detected.

### Example 2—Pairing of CD3 Heavy Chains with the Light Chains of Several Commercial or Publicly Available Antibodies

Essentially, the study of Example 1 was repeated with the light chains of the commercial or publicly available antibodies listed in Table 3.

TABLE 3
Commercial or publicly available antibodies, the
light chain variable regions (or sequence variants
thereof) of which were used in this study.

| Commercial or publicly | | Light chain variable |
|---|---|---|
| available antibody | Target | region sequence |
| Urelumab | CD137 | SEQ ID NO: 43 or 68 |
| Milatuzumab | CD74 | SEQ ID NO: 44 |
| Cusatuzumab | CD70 | SEQ ID NO: 45 or 69 |
| Belantamab | BCMA | SEQ ID NO: 46 |
| Daratumumab | CD38 | SEQ ID NO: 47 |
| Epratuzumab | CD22 | SEQ ID NO: 48 |
| Fresolimumab | TGF-β | SEQ ID NO: 49 |
| Inotuzumab | CD22 | SEQ ID NO: 50 |
| KA3 | CD47 | SEQ ID NO: 51 |
| Lemzoparlimab | CD47 | SEQ ID NO: 52 |
| Magrolimab | CD47 | SEQ ID NO: 53 |
| Monalizumab | NKG2A | SEQ ID NO: 54 |
| Obinutuzumab | CD20 | SEQ ID NO: 55 |
| Ofatumumab | CD20 | SEQ ID NO: 56 |
| Olaratumab | PDGFRA | SEQ ID NO: 57 |
| Omburtamab | B7-H3 | SEQ ID NO: 58 or 70 |
| Gatipotuzumab (PankoMab) | MUC-1 | SEQ ID NO: 59 or 71 |
| Plozalizumab | CCR2 | SEQ ID NO: 60 |
| Rituximab | CD20 | SEQ ID NO: 61 |
| Sibrotuzumab | FAP | SEQ ID NO: 62 |
| Tafasitamab | CD19 | SEQ ID NO: 63 |
| Tovetumab | PDGFRA | SEQ ID NO: 64 or 72 |
| Ulocuplumab | CXCR4 | SEQ ID NO: 65 |
| H2C | SIGLEC15 | SEQ ID NO: 66 |
| Teplizumab (hOKT3) | CD3 | SEQ ID NO: 67 |
| Trastuzumab | HER2 | SEQ ID NO: 41 |
| Vκ1-39/Jκ1 cLC | — | SEQ ID NO: 5 |

### ELISA

The CD3 antibodies were screened in order to determine whether the antibodies are capable of specific binding to CD3. ELISA plates (Greiner Bio-One, Cat. No. 655061) were coated with CD388-FC or tetanus toxoid (TT) (AJ Vaccines, Cat. No. 2674) at 2.5 μg/ml and 2 μg/ml in PBS, respectively. Coated plates were incubated overnight at 4° C., washed twice in freshly prepared wash buffer (0.05% Tween 20; Merck, Cat. No. 8.22184.0500, prepared in PBS) and blocked for 1 hour at RT with blocking buffer (2% BSA; Sigma, Cat. No. A3294-500g, prepared in PBS). CD3 antibodies or control antibodies diluted in blocking buffer were added to the wells at a concentration of 5 μg/ml. Antibodies were incubated for 1 hour at RT. Control antibodies include: positive control anti-CD3 antibody used to confirm coating of the CD3 antigen and negative control IgG1 antibody (TT) used to confirm specificity of binding. Wells were washed three times in wash buffer before the addition of the secondary antibodies. Anti-hu IgG HRP antibody (BD, Cat. No. 555788) diluted at 1:2000 in blocking buffer was added to wells for 1 hour incubation at RT. Wells were washed three times in washing buffer before development. 50 μl of TMB substrate solution, prepared freshly by mixing 1× reagent A:1× reagent B (BD OptEIA TMB Substrate Reagent Set from BD, Cat. No. 555214), was added to each well for maximal 10 minutes before the reaction was stopped with 50 ul of 1M H₂SO₄ (Merck, Cat. No. 1.00731). BioTek Elx808 ELISA plate reader was used for the read out at O.D. 450 nm and titration data was analyzed using GraphPad prism software.

Antibodies that showed more than three times background binding to CD3 and less than three times background binding to TT were selected for further FACS analysis.

### FACS

Binding and relative affinity of the CD3 antibodies to huCD3 was determined by FACS analysis on HPB-ALL cells. HPB-ALL cells were cultured in RPMI 1640 (Thermo Fisher, Cat. No. 21875) with 10% heat inactivated (h.i.) fetal bovine serum (FBS) (Sigma, Cat. No. F7524) seeded at 0.05-0.2×10⁶ cells/well. To block Fc function, cells were incubated with FACS blocking buffer (0.5% BSA, Sigma-Aldrich, Cat. No. A3294 and 2 mM EDTA, Invitrogen, Cat. No. 15575-020+ 3% Rabbit Serum, Sigma-Aldrich, Cat. No. R9133). CD3 antibodies, positive control anti-CD3 antibody or negative control IgG1 antibody (TT) were prepared in FACS buffer (0.5% BSA, Sigma-Aldrich, Cat. No. A3294 and 2 mM EDTA, Invitrogen, Cat. No. 15575-020) and added to the cells at an eight-step semi-log serial dilutions starting at 10 μg/ml. Cells were incubated for 30 minutes and washed before the addition of anti-hu IgG R-PE secondary antibody (Invitrogen, Cat. No. H10104) at 1:100 for 30 minutes incubation and washing, followed by FACS analysis. All incubation and washing steps were done in ice-cold FACS buffer. FACS measurements were performed using iQue VBR; Intellicyt and ForeCyt software to represent the mean fluorescence intensity (MFI).

For the data analysis, all binding curves were plotted grouped per VH and AUC were calculated (GraphPad prism software). AUCs of the VHs with the commercial or publicly available antibody light chains were compared to the same VHs combined with their original light chain.

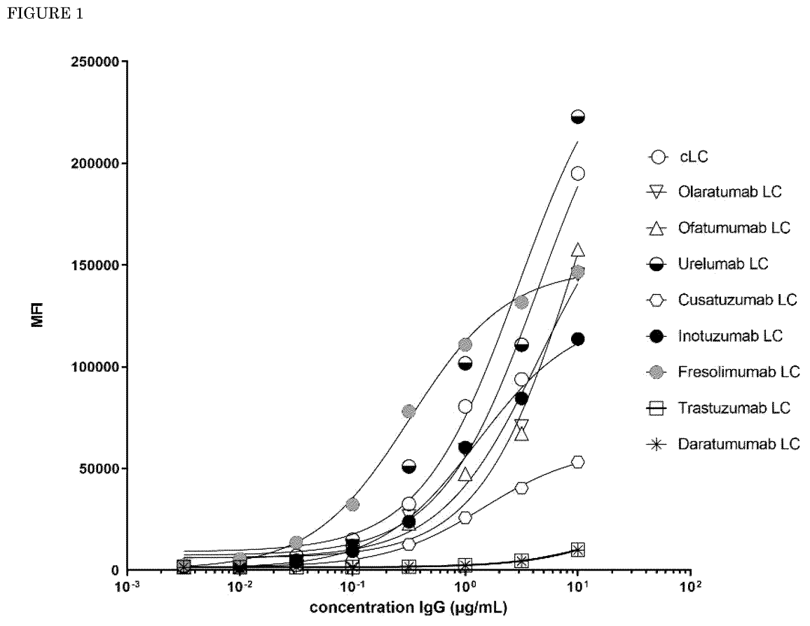

On overview of the results of the ELISA is shown in Table 4. These data show that antibody SC5Ab1 has the most promiscuous VH. The FACS data for this VH is provided in FIG. 1.

### Example 3—Further Characterization of Antibodies Comprising the Heavy Chain Variable Region of Antibody SC5Ab1 and the Light Chain Variable Region of Several Commercial or Publicly Available Antibodies

The heavy chain variable region (VH) of antibody SC5Ab1 was combined with the light chain variable regions (VL) of the commercial or publicly available antibodies cusatuzumab, fresolimumab, ofatumumab, and trastuzumab. The VH region of antibody SC5Ab1 was also combined with the VL region having an amino acid sequence as set forth in SEQ ID NO: 5, to serve as parental control.

In order to determine the molecular weight sizing and percent purity of the IgGs, IgG samples were analyzed by Labchip (LabChip GXII Touch HT; Perkin Elmer) under non-reducing conditions using Protein Clear HR Reagent kit (Dye Solution, Sample Buffer, Protein Gel Matrix, Protein Ladder, Lower Marker, Wash Buffer; Perkin Elmer CLS960014) and Protein Express Assay LabChip for use with GXII Touch HT (Elmer 760499) according to the manufacturer's instructions; Labchip RX reviewer software was used for protein characterization. Two hundred and fifty nanograms of each sample were analyzed.

The binding capacity of the IgGs was tested on HPB-ALL cells, as described in Example 2, with the difference that here the negative control was an IgG1 antibody targeting RSV-G.

The melting temperature (Tm) and aggregation temperature (Tagg) of the non-cLC IgGs and the corresponding parental cLC IgG were measured using an Uncle system (Unchained Labs, product code 200-1037). Tm was measured by the change of intrinsic fluorescence of amino acid upon heating at a spectral range between 250 and 720 nm, and simultaneously, Tagg was measured by static light scattering (SLS) at 266 nm. Solutions of IgG samples were prepared in PBS PH 7.4 (1×, Gibco, Cat. No. 10010-031) at final concentration 100 μg/mL, or 250 μg/ml for assay controls. 8.8 μl of IgG solutions or PBS were loaded into the microcuvettes (Unis, product code 201-1009). Assembly of the Uni, seals (product code 201-1009/201-1013) and uni frames (product code 201-1012), and loading into UNcle system was performed according to manufacturer's instructions. IgG samples were heated from 25 to 95° C. at 0.3° C./min with an equilibration time of 1 min before thermal ramp. UNcle analysis v5.01 software was used for data analysis and generation of melting and aggregation curves.

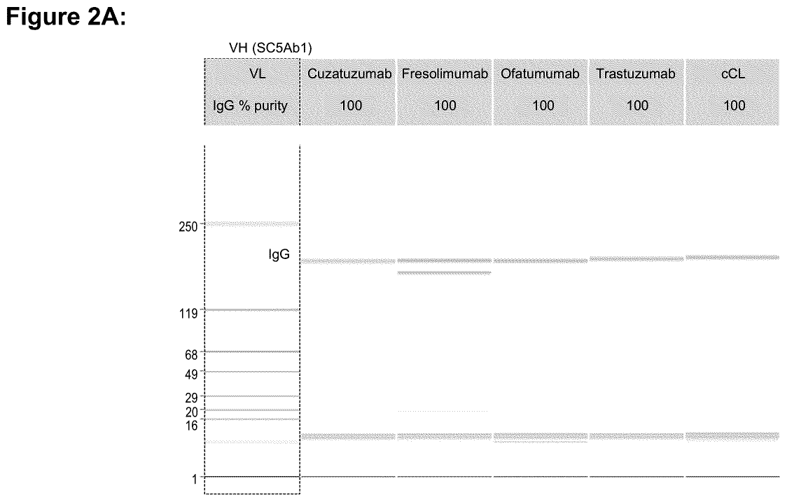

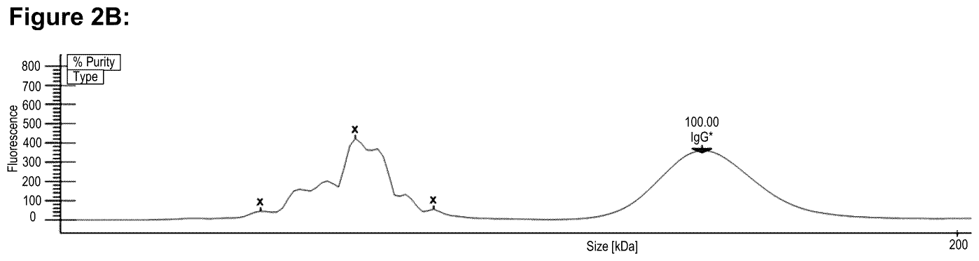

The data, as shown in FIG. 2A, shows that the heavy chain variable region (VH) of antibody SC5Ab1 pairs well with multiple, non-cognate, light chain variable regions, as demonstrated by a major band corresponding to the IgG (150 kD). Manual inspection of the individual chromatogram revealed that the additional bands, for example as seen in the case of fresolimumab, do not correspond to protein peaks (FIG. 2B).

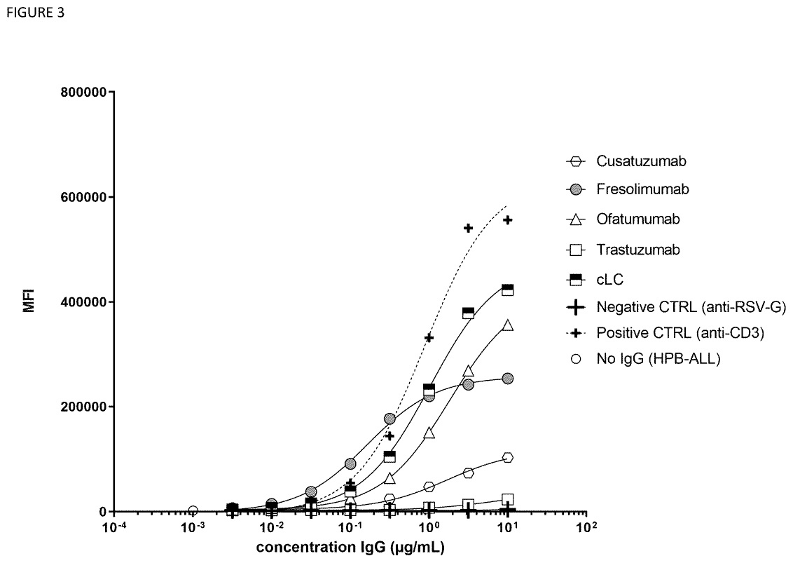

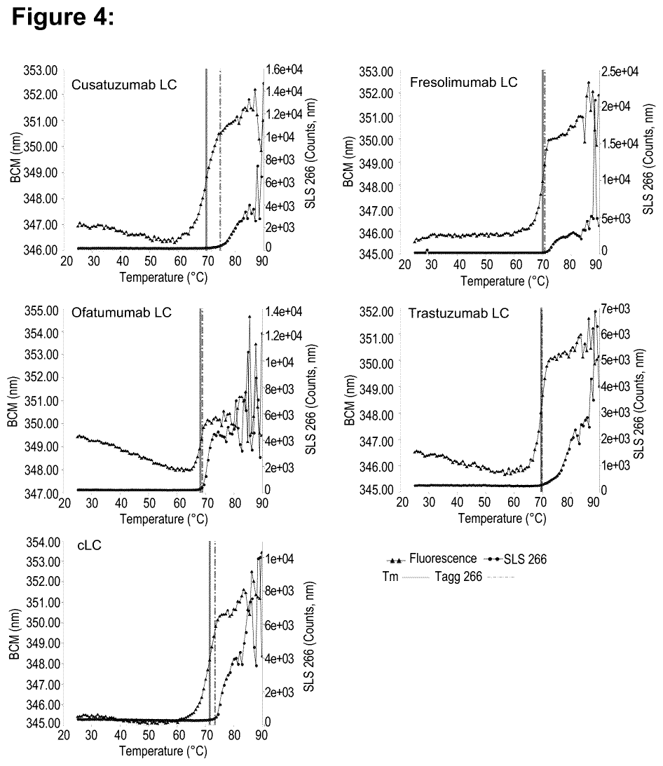

In conclusion, antibodies comprising the VH region of antibody SC5Ab1 paired with the VL region of most of the commercial or publicly available antibodies retain adequate CD3 binding (FIG. 3) and show a good developability profile, having a thermostability comparable to when paired with its cognate VL region (SEQ ID NO: 5) (see FIG. 4).

TABLE 4
Results of the ELISA.
Binding fold over background binding to CD3 is depicted. N.A. Not available.

| | CD3 antibody | | | | | | | |
|---|---|---|---|---|---|---|---|---|
| VL | SC1Ab2 | SC2Ab1 | SC3Ab2 | SC4Ab1 | SC5Ab1 | SC6Ab1 | SC7Ab2 | SC8Ab1 |
| Urelumab | 1.04 | 2.31 | 1.33 | 1.00 | 24.75 | 1.47 | 8.55 | 1.16 |
| MilatuzuMab | N.A. | 1.04 | 1.00 | N.A. | N.A. | 1.00 | N.A. | N.A. |
| Cusatuzumab | 1.14 | 1.06 | 1.06 | 1.02 | 22.02 | 1.06 | 1.76 | 1.02 |
| Belantamab | 0.94 | 21.24 | 20.59 | 1.06 | 3.63 | 1.55 | 1.02 | 1.12 |
| Daratumumab | 1.02 | 6.04 | 9.35 | 0.94 | 8.04 | 1.35 | 1.00 | 1.12 |
| Epratuzumab | 1.33 | 1.04 | 0.98 | N.A. | 1.75 | N.A. | N.A. | 1.20 |
| Fresolimumab | 1.02 | 1.06 | 1.16 | 2.69 | 22.47 | 1.04 | 1.08 | 1.61 |
| Inotuzumab | 1.00 | 1.43 | 1.04 | 0.96 | 23.14 | 1.00 | 18.06 | 1.06 |
| KA3 | 1.02 | 1.06 | 0.98 | 1.06 | N.A. | 1.18 | N.A. | N.A. |
| Lemzoparlimab | 0.94 | 1.49 | 9.24 | 0.96 | 22.31 | 0.96 | N.A. | 1.10 |
| Magrolimab | 1.10 | 1.10 | 1.04 | 0.98 | 23.96 | 0.96 | N.A. | 1.04 |
| Monalizumab | 0.96 | 1.04 | 19.80 | 1.08 | N.A. | 1.53 | N.A. | 1.04 |
| Obinutuzumab | 1.02 | 1.08 | 1.00 | 0.98 | N.A. | 0.98 | N.A. | 1.14 |
| Ofatumumab | 1.06 | 3.06 | 2.55 | 1.08 | 24.22 | N.A. | 1.12 | 1.16 |
| Olaratumab | 1.00 | 4.12 | 4.41 | 0.96 | 23.88 | N.A. | 1.10 | 1.31 |
| Omburtamab | 1.14 | 1.10 | 5.43 | 0.96 | 21.53 | 0.96 | 1.02 | 1.02 |
| Gatipotuzumab | 0.02 | 1.33 | 1.80 | 1.18 | 15.00 | 1.08 | N.A. | 1.14 |
| (PankoMab) | | | | | | | | |
| Plozalizumab | 0.98 | 1.18 | N.A. | 0.96 | N.A. | 0.94 | N.A. | 1.04 |
| Rituximab | 1.20 | 1.14 | 1.06 | 1.00 | 2.75 | 0.94 | 1.06 | 1.00 |
| Sibrotuzumab | 1.14 | 1.06 | 1.94 | 1.35 | 5.57 | 0.98 | N.A. | 1.04 |
| Tafasitamab | 1.12 | 1.10 | 1.00 | 1.00 | N.A. | 0.98 | N.A. | 1.12 |
| Tovetumab | 1.33 | 1.06 | 1.59 | 26.55 | 23.29 | 1.78 | N.A. | N.A. |
| Ulocuplumab | N.A. | 1.02 | 1.00 | 1.04 | N.A. | 1.02 | N.A. | N.A. |
| H2C | 1.04 | 1.00 | 0.94 | 1.02 | 9.92 | 1.00 | N.A. | 1.24 |
| Teplizumab | N.A. | 1.25 | 1.04 | N.A. | N.A. | 1.02 | N.A. | N.A. |
| (hOKT3) | | | | | | | | |
| Trastuzumab | 1.00 | 1.00 | 1.04 | 0.98 | 13.39 | 1.22 | 1.02 | 1.00 |
| cLC | 23.27 | 22.27 | 19.76 | 25.16 | 22.37 | N.A. | 24.41 | 21.12 |

SEQUENCES

SEQ ID NO: 1-Heavy chain variable region
EVQLVESGAEVKKPGESLKISCKASGYSFTRFYIGWVRQMPGKGLEWMGLIYPGDS
DTKYSPSFQGQVTISVDKSINTAYLQWNSLKASDTAMYYCARHIRYFDWFSHYYYM
DVWGKGTMVTVSS
SEQ ID NO: 2-Heavy chain CDR1 according to Kabat
RFYIG
SEQ ID NO: 3-Heavy chain CDR2 according to Kabat
LIYPGDSDTKYSPSFQG
SEQ ID NO: 4-Heavy chain CDR3 according to Kabat
HIRYFDWFSHYYYMDV
SEQ ID NO: 5-Light chain variable region cLC
DIQMTQSPSSLSASVGDRVTITCRASQSISSYLNWYQQKPGKAPKLLIYAASSLQSGVP
SRFSGSGSGTDFTLTISSLQPEDFATYYCQQSYSTPPTFGQGTKVEIK
SEQ ID NO: 6-Light chain CDR1 according to IMGT
QSISSY
SEQ ID NO: 7-Light chain CDR2 according to IMGT
AAS
SEQ ID NO: 8-Light chain CDR3 according to IMGT
QQSYSTPPT
SEQ ID NO: 9-CL region
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID NO: 10-Light chain cLC
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPPTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 11-V region VK1-39
DIQMTQSPSSLSASVGDRVTITCRASQSISSYLNWYQQKPGKAPKLLIYAASSLQSGV
PSRFSGSGSGTDFTLTISSLQPEDFATYYCQQSYSTP
SEQ ID NO: 12-VK1-39/JK1
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPPTFGQGTKVEIK
SEQ ID NO: 13-Light chain variable region
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPPITFGQGTRLEIK
SEQ ID NO: 14-Light chain CDR1 according to IMGT
QSISSY
SEQ ID NO: 15-Light chain CDR2 according to IMGT
AAS
SEQ ID NO: 16-Light chain CDR3 according to IMGT
QQSYSTPPIT
SEQ ID NO: 17-V region VK3-15
EIVMTQSPATLSVSPGERATLSCRASQSVSSNLAWYQQKPGQAPRLLIYGASTRATGI
PARFSGSGSGTEFTLTISSLQSEDFAVYYCQQYNNWP
SEQ ID NO: 18-Light chain variable region
EIVMTQSPATLSVSPGERATLSCRASQSVSSNLAWYQQKPGQAPRLLIYGASTRATGI
PARFSGSGSGTEFTLTISSLQSEDFAVYYCQQYNNWPWTFGQGTKVEIK
SEQ ID NO: 19-Light chain CDR1 according to IMGT
QSVSSN
SEQ ID NO: 20-Light chain CDR2 according to IMGT
GAS
SEQ ID NO: 21-Light chain CDR3 according to IMGT
QQYNNWPWT
SEQ ID NO: 22-V region VK3-20
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSP
SEQ ID NO: 23-Light chain variable region
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSPWTFGQGTKVEIK
SEQ ID NO: 24-Light chain CDR1 according to IMGT
QSVSSSY
SEQ ID NO: 25-Light chain CDR2 according to IMGT
GAS
SEQ ID NO: 26-Light chain CDR3 according to IMGT
QQYGSSPWT
SEQ ID NO: 27-V region VL3-21
SYVLTQPPSVSVAPGETARITCGGDNIGRKSVYWYQQKSGQAPVLVIYYDSDRPSGIP
ERFSGSNSGNTATLTISRVEAGDEADYYCQVWDGSSDH
SEQ ID NO: 28-Light chain variable region
SYVLTQPPSVSVAPGETARITCGGDNIGRKSVYWYQQKSGQAPVLVIYYDSDRPSGIP
ERFSGSNSGNTATLTISRVEAGDEADYYCQVWDGSSDHWVFGGGTKLTVL
SEQ ID NO: 29-Light chain CDR1 according to IMGT
NIGRKS
SEQ ID NO: 30-Light chain CDR2 according to IMGT
YDS
SEQ ID NO: 31-Light chain CDR3 according to IMGT
QVWDGSSDHWV
SEQ ID NO: 32-hinge region
EPKSCDKTHTCPPCP
SEQ ID NO: 33-CH1 region
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRV
SEQ ID NO: 34-CH2 region
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAK
SEQ ID NO: 35-CH2-DM region
APELGRGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAK
SEQ ID NO: 36-CH3 region
GQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
SEQ ID NO: 37-Heavy chain positive control anti-CD3 antibody
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSL
SPG
SEQ ID NO: 38-Heavy chain negative control IgG1 antibody (RSV-G)
EVQLVESGGGVVQPGRSLRLSCAASGFTFSNYGMHWVRQAPGKGLEWVAVISYDG
STKYSADSLKGRFTISRDNSKNTLYLQMNSLRADDTAVYYCAKEGWSFDSSGYRSW
FDSWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLS
PG
SEQ ID NO: 39-Heavy chain negative control IgG1 antibody (TT)
EVQLVETGAEVKKPGASVKVSCKASDYIFTKYDINWVRQAPGQGLEWMGWMSAN
TGNTGYAQKFQGRVTMTRDTSINTAYMELSSLTSGDTAVYFCARSSLFKTETAPYYH
FALDVWGQGTTVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVE
PKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLS
LSPG
SEQ ID NO: 40-Light chain variable region cetuximab
DILLTQSPVILSVSPGERVSFSCRASQSIGTNIHWYQQRINGSPRLLIKYASESISGIPSR
FSGSGSGTDFTLSINSVESEDIADYYCQQNNNWPTTFGAGTKLELK
SEQ ID NO: 41-Light chain variable region trastuzumab
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIK
SEQ ID NO: 42-Light chain variable region nivolumab
EIVLTQSPATLSLSPGERATLSCRASQSVSSYLAWYQQKPGQAPRLLIYDASNRATGIP
ARFSGSGSGTDFTLTISSLEPEDFAVYYCQQSSNWPRTFGQGTKVEIK
SEQ ID NO: 43-Light chain variable region urelumab
EIVLTQSPATLSLSPGERATLSCRASQSVSSYLAWYQQKPGQAPRLLIYDASNRATGIP
ARFSGSGSGTDFTLTISSLEPEDFAVYYCQQRSNWPPALTFGGGTKVEIK
SEQ ID NO: 44-Light chain variable region milatuzumab
DIQLTQSPLSLPVTLGQPASISCRSSQSLVHRNGNTYLHWFQQRPGQSPRLLIYTVSNR
FSGVPDRFSGSGSGTDFTLKISRVEAEDVGVYFCSQSSHVPPTFGAGTRLEIK
SEQ ID NO: 45-Light chain variable region cusatuzumab
QAVVTQEPSLTVSPGGTVTLTCGLKSGSVTSDNFPTWYQQTPGQAPRLLIYNTNTRHS
GVPDRFSGSILGNKAALTITGAQADDEAEYFCALFISNPSVEFGGGTQLEIK
SEQ ID NO: 46-Light chain variable region belantamab
DIQMTQSPSSLSASVGDRVTITCSASQDISNYLNWYQQKPGKAPKLLIYYTSNLHSGV
PSRFSGSGSGTDFTLTISSLQPEDFATYYCQQYRKLPWTFGQGTKLEIK
SEQ ID NO: 47-Light chain variable region daratumumab
EIVLTQSPATLSLSPGERATLSCRASQSVSSYLAWYQQKPGQAPRLLIYDASNRATGI
PARFSGSGSGTDFTLTISSLEPEDFAVYYCQQRSNWPPTFGQGTKVEIK
SEQ ID NO: 48-Light chain variable region epratuzumab
DIQLTQSPSSLSASVGDRVTMSCKSSQSVLYSANHKNYLAWYQQKPGKAPKLLIYW
ASTRESGVPSRFSGSGSGTDFTFTISSLQPEDIATYYCHQYLSSWTFGGGTKLEIK
SEQ ID NO: 49-Light chain variable region fresolimumab
ETVLTQSPGTLSLSPGERATLSCRASQSLGSSYLAWYQQKPGQAPRLLIYGASSRAPGI
PDRFSGSGSGTDFTLTISRLEPEDFAVYYCQQYADSPITFGQGTRLEIK
SEQ ID NO: 50-Light chain variable region inotuzumab
DVQVTQSPSSLSASVGDRVTITCRSSQSLANSYGNTFLSWYLHKPGKAPQLLIYGISNR
FSGVPDRFSGSGSGTDFTLTISSLQPEDFATYYCLQGTHQPYTFGQGTKVEIK
SEQ ID NO: 51-Light chain variable region KA3
DIQMTQSPSSLSASVGDRVTITCRASQSISSYLNWYQQKPGKAPKLLIYAASSLQSGV
PSRFSGSGSGTDFTLTISSLQPEDFATYYCQQMHPRAPKTFGQGTKVEIK
SEQ ID NO: 52-Light chain variable region lemzoparlimab
DIVMTQSPDSLAVSLGERATINCKSSQSVLYAGNNRNYLAWYQQKPGQPPKLLINQAS
TRASGVPDRFSGSGSGTEFTLIISSLQAEDVAIYYCQQYYTPPLAFGGGTKLEIK
SEQ ID NO: 53-Light chain variable region magrolimab
DIVMTQSPLSLPVTPGEPASISCRSSQSIVYSNGNTYLGWYLQKPGQSPQLLIYKVSNRF
SGVPDRFSGSGSGTDFTLKISRVEAEDVGVYYCFQGSHVPYTFGQGTKLEIK
SEQ ID NO: 54-Light chain variable region monalizumab
DIQMTQSPSSLSASVGDRVTITCRASENIYSYLAWYQQKPGKAPKLLIYNAKTLAEG
VPSRFSGSGSGTDFTLTISSLQPEDFATYYCQHHYGTPRTFGGGTKVEIK
SEQ ID NO: 55-Light chain variable region obinutuzumab
DIVMTQTPLSLPVTPGEPASISCRSSKSLLHSNGITYLYWYLQKPGQSPQLLIYQMSNL
VSGVPDRFSGSGSGTDFTLKISRVEAEDVGVYYCAQNLELPYTFGGGTKVEIK
SEQ ID NO: 56-Light chain variable region ofatumumab
EIVLTQSPATLSLSPGERATLSCRASQSVSSYLAWYQQKPGQAPRLLIYDASNRATGIP
ARFSGSGSGTDFTLTISSLEPEDFAVYYCQQRSNWPITFGQGTRLEIK
SEQ ID NO: 57-Light chain variable region olaratumab
EIVLTQSPATLSLSPGERATLSCRASQSVSSYLAWYQQKPGQAPRLLIYDASNRATGIP
ARFSGSGSGTDFTLTISSLEPEDFAVYYCQQRSNWPPAFGQGTKVEIK
SEQ ID NO: 58-Light chain variable region omburtamab
EIVMTQSPATLSVSPGERVSLSCRASQSISDYLHWYQQKSHESPRLLIKYASQSISGIPA
RFSGSGSGSEFTLTINSVEPEDVGVYYCQNGHSFPLTFGQGTKLEIK
SEQ ID NO: 59-Light chain variable region pankomab
DIVMTQAAFSNPVTLGTSASISCRSSKSLLHSNGITYFFWYLQKPGLSPQLLIYQMSNLA
SGVPDRFSSSGSGTDFTLRISRVEAEDVGVYYCAQNLELPPTFGGGTKLEIK
SEQ ID NO: 60-Light chain variable region plozalizumab
DVVMTQSPLSLPVTLGQPASISCKSSQSLLDSDGKTFLNWFQQRPGQSPRRLIYLVSK
LDSGVPDRFSGSGSGTDFTLKISR VEAEDVGVYYCWQGTHEPYTFGQGTRLEIK
SEQ ID NO: 61-Light chain variable region rituximab
QIVLSQSPAILSASPGEKVTMTCRASSSVSYIHWFQQKPGSSPKPWIYATSNLASGVP
VRFSGSGSGTSYSLTISRVEAEDAATYYCQQWTSNPPTFGGGTKLEIK
SEQ ID NO: 62-Light chain variable region sibrotuzumab
DIVMTQSPDSLAVSLGERATINCKSSQSLLYSRNQKNYLAWYQQKPGQPPKLLIFWA
STRESGVPDRFSGSGFGTDFTLTISSLQAEDVAVYYCQQYFSYPLTFGQGTKVEIK
SEQ ID NO: 63-Light chain variable region tafasitamab
DIVMTQSPATLSLSPGERATLSCRSSKSLQNVNGNTYLYWFQQKPGQSPQLLIYRMS
NLNSGVPDRFSGSGSGTEFTLTISSLEPEDFAVYYCMQHLEYPITFGAGTKLEIK
SEQ ID NO: 64-Light chain variable region tovetumab
DIQMTQSPSSLSASVGDRVSITCRPSQSFSRYINWYQQKPGKAPKLLIHAASSLVGGVP
SRFSGSGSGTDFTLTISSLQPEDFATYYCQQTYSNPPITFGQGTRLEIK
SEQ ID NO: 65-Light chain variable region ulocuplumab
DIQMTQSPSSLSASVGDRVTITCRASQGISSWLAWYQQKPEKAPKSLIYAASSLQSGV
PSRFSGSGSGTDFTLTISSLQPEDFVTYYCQQYNSYPRTFGQGTK VEIK
SEQ ID NO: 66-Light chain variable region H2C
QTVVTQEPSLTVSPGGTVTLTCGSSTGAVTSGYYPNWVQQKPGQAPRGLIGGTKFLA
PGTPARFSGSLLGGKAALTLSGVQPEDEAEYYCALWYSNRWVFGGGTKLTVL
SEQ ID NO: 67-Light chain variable region hOKT3
DIQMTQSPSSLSASVGDRVTITCSASSSVSYMNWYQQTPGKAPKRWIYDTSKLASGV
PSRFSGSGSGTDYTFTISSLQPEDIATYYCQQWSSNPFTFGQGTKLQIT
SEQ ID NO: 68-Light chain variable region
EIVLTQSPATLSLSPGERATLSCRASQSVSSYLAWYQQKPGQAPRLLIYDASNRATGIP
ARFSGSGSGTDFTLTISSLEPEDFAVYYCQQRSNWPPALTFCGGTKVEIK
SEQ ID NO: 69-Light chain variable region
QAVVTQEPSLTVSPGGTVTLTCGLKSGSVTSDNFPTWYQQTPGQAPRLLIYNTNTRHS
GVPDRFSGSILGNKAALTITGAQADDEAEYFCALFISNPSVEFGGGTQLTVL
SEQ ID NO: 70-Light chain variable region
DIVMTQSPATLSVTPGDRVSLSCRASQSISDYLHWYQQKSHESPRLLIKYASQSISGIPS
RFSGSGSGSDFTLSINSVEPEDVGVYYCQNGHSFPLTFGAGTKLELK
SEQ ID NO: 71-Light chain variable region
DIVMTQSPLSNPVTPGEPASISCRSSKSLLHSNGITYFFWYLQKPGQSPQLLIYQMSNLA
SGVPDRFSGSGSGTDFTLRISRVEAEDVGVYYCAQNLELPPTFGQGTKVEIK
SEQ ID NO: 72-Light chain variable region
DIQMTQSPSSLSASVGDRVSITCRPSQSFSRYINWYQQKPGKAPKLLIHAASSLVGGVP
SRFSGSGSGTDFTLTISSLQPEDFATYYCQQTYSNPPITFGQGTRLEMK
SEQ ID NO: 73-Light chain CDR1 according to IMGT
QDVNTA
SEQ ID NO: 74-Light chain CDR2 according to IMGT
SAS
SEQ ID NO: 75-Light chain CDR3 according to IMGT
QQHYTTPPT
SEQ ID NO: 76-Light chain CDR1 according to IMGT
QSVSSY
SEQ ID NO: 77-Light chain CDR2 according to IMGT
DAS
SEQ ID NO: 78-Light chain CDR3 according to IMGT
QQSSNWPRT
SEQ ID NO: 79-Light chain CDR1 according to IMGT
QSVSSY
SEQ ID NO: 80-Light chain CDR2 according to IMGT
DAS
SEQ ID NO: 81-Light chain CDR3 according to IMGT
QQRSNWPPALT
SEQ ID NO: 82-Light chain CDR3 according to IMGT
QQRSNWPPALTFC
SEQ ID NO: 83-Light chain CDR1 according to IMGT
SGSVTSDNF
SEQ ID NO: 84-Light chain CDR2 according to IMGT
NTN
SEQ ID NO: 85-Light chain CDR3 according to IMGT
ALFISNPSVE
SEQ ID NO: 86-Light chain CDR1 according to IMGT
QSLGSSY
SEQ ID NO: 87-Light chain CDR2 according to IMGT
GAS
SEQ ID NO: 88-Light chain CDR3 according to IMGT
QQYADSPIT
SEQ ID NO: 89-Light chain CDR1 according to IMGT
QSLANSYGNTF
SEQ ID NO: 90-Light chain CDR2 according to IMGT
GIS
SEQ ID NO: 91-Light chain CDR3 according to IMGT
LQGTHQPYT
SEQ ID NO: 92-Light chain CDR1 according to IMGT
QSVLYAGNNRNY
SEQ ID NO: 93-Light chain CDR2 according to IMGT
QAS
SEQ ID NO: 94-Light chain CDR3 according to IMGT
QQYYTPPLA
SEQ ID NO: 95-Light chain CDR1 according to IMGT
QSIVYSNGNTY
SEQ ID NO: 96-Light chain CDR2 according to IMGT
KVS
SEQ ID NO: 97-Light chain CDR3 according to IMGT
FQGSHVPYT
SEQ ID NO: 98-Light chain CDR1 according to IMGT
QSVSSY
SEQ ID NO: 99-Light chain CDR2 according to IMGT
DAS
SEQ ID NO: 100-Light chain CDR3 according to IMGT
QQRSNWPIT
SEQ ID NO: 101-Light chain CDR1 according to IMGT
QSVSSY
SEQ ID NO: 102-Light chain CDR2 according to IMGT
DAS
SEQ ID NO: 103-Light chain CDR3 according to IMGT
QQRSNWPPA
SEQ ID NO: 104-Light chain CDR1 according to IMGT
QSISDY
SEQ ID NO: 105-Light chain CDR2 according to IMGT
YAS
SEQ ID NO: 106-Light chain CDR3 according to IMGT
QNGHSFPLT
SEQ ID NO: 107-Light chain CDR1 according to IMGT
KSLLHSNGITY
SEQ ID NO: 108-Light chain CDR2 according to IMGT
QMS
SEQ ID NO: 109-Light chain CDR3 according to IMGT
AQNLELPPT
SEQ ID NO: 110-Light chain CDR1 according to IMGT
QSFSRY
SEQ ID NO: 111-Light chain CDR2 according to IMGT
AAS
SEQ ID NO: 112-Light chain CDR3 according to IMGT
QQTYSNPPIT
SEQ ID NO: 113-Human CD3d
MEHSTFLSGLVLATLLSQVSPFKIPIEELEDRVFVNCNTSITWVEGTVGTLLSDITRLD
LGKRILDPRGIYRCNGTDIYKDKESTVQVHYRMCQSCVELD
SEQ ID NO: 114-Human CD3e
MQSGTHWRVLGLCLLSVGVWGQDGNEEMGGITQTPYKVSISGTTVILTCPQYPGSEI
LWQHNDKNIGGDEDDKNIGSDEDHLSLKEFSELEQSGYYVCYPRGSKPEDANFYLY
LRARVCENCMEMD

1. A polypeptide comprising a heavy chain CDR3 (HCDR3) having an amino acid sequence as set forth in SEQ ID NO: 4; or having at least 70% sequence identity thereto.

2. The polypeptide according to claim 1, wherein the polypeptide comprises a heavy chain CDR1 (HCDR1) having an amino acid sequence as set forth in SEQ ID NO: 2, a heavy chain CDR2 (HCDR2) having an amino acid sequence as set forth in SEQ ID NO: 3, and a heavy chain CDR3 (HCDR3) having an amino acid sequence as set forth in SEQ ID NO: 4; or having at least 70% sequence identity to said HCDR3.

3. The polypeptide according to claim 1 or 2, wherein the polypeptide comprises a heavy chain CDR1 (HCDR1) having an amino acid sequence as set forth in SEQ ID NO: 2, a heavy chain CDR2 (HCDR2) having an amino acid sequence as set forth in SEQ ID NO: 3, and a heavy chain CDR3 (HCDR3) having an amino acid sequence as set forth in SEQ ID NO: 4,
wherein each of the HCDRs may contain one, two, or at most three amino acid variations.

4. The polypeptide according to any one of claims 1-3, wherein the polypeptide comprises a heavy chain variable region having an amino acid sequence as set forth in SEQ ID NO: 1, or having at least 80%, or at least 85%, or at least 90%, or at least 95%, sequence identity thereto.

5. The polypeptide according to any one of claims 1-4, further comprising a CH1 region.

6. The polypeptide according to any one of claims 1-5, further comprising a hinge, CH2 region, and CH3 region.

7. A CD3 binding domain comprising the polypeptide according to any one of claims 1-6.

8. The CD3 binding domain according to claim 7, wherein the CD3 binding domain further comprises a polypeptide comprising a light chain variable region.

9. A group of antigen-binding proteins that bind human CD3, wherein each antigen-binding protein within the group comprises a heavy chain variable region and light chain variable region, the heavy chain variable region comprising a HCDR3 having at least 70% sequence identity to the amino acid sequence as set forth in SEQ ID NO: 4.

10. The CD3 binding domain according to claim 8 or the group of antigen-binding proteins according to claim 9, wherein the light chain variable region comprises:
the light chain CDR1 (LCDR1), light chain CDR2 (LCDR2), and light chain CDR3 (LCDR3), of a light chain variable region having an amino acid sequence as set forth in SEQ ID NO: 5;
the light chain CDR1 (LCDR1), light chain CDR2 (LCDR2), and light chain CDR3 (LCDR3), of a light chain variable region having an amino acid sequence as set forth in SEQ ID NO: 41;
the light chain CDR1 (LCDR1), light chain CDR2 (LCDR2), and light chain CDR3 (LCDR3), of a light chain variable region having an amino acid sequence as set forth in SEQ ID NO: 42;
the light chain CDR1 (LCDR1), light chain CDR2 (LCDR2), and light chain CDR3 (LCDR3), of a light chain variable region having an amino acid sequence as set forth in SEQ ID NO: 43 or 68;
the light chain CDR1 (LCDR1), light chain CDR2 (LCDR2), and light chain CDR3 (LCDR3), of a light chain variable region having an amino acid sequence as set forth in SEQ ID NO: 45 or 69;
the light chain CDR1 (LCDR1), light chain CDR2 (LCDR2), and light chain CDR3 (LCDR3), of a light chain variable region having an amino acid sequence as set forth in SEQ ID NO: 49;
the light chain CDR1 (LCDR1), light chain CDR2 (LCDR2), and light chain CDR3 (LCDR3), of a light chain variable region having an amino acid sequence as set forth in SEQ ID NO: 50;
the light chain CDR1 (LCDR1), light chain CDR2 (LCDR2), and light chain CDR3 (LCDR3), of a light chain variable region having an amino acid sequence as set forth in SEQ ID NO: 52;
the light chain CDR1 (LCDR1), light chain CDR2 (LCDR2), and light chain CDR3 (LCDR3), of a light chain variable region having an amino acid sequence as set forth in SEQ ID NO: 53;
the light chain CDR1 (LCDR1), light chain CDR2 (LCDR2), and light chain CDR3 (LCDR3), of a light chain variable region having an amino acid sequence as set forth in SEQ ID NO: 56;
the light chain CDR1 (LCDR1), light chain CDR2 (LCDR2), and light chain CDR3 (LCDR3), of a light chain variable region having an amino acid sequence as set forth in SEQ ID NO: 57;
the light chain CDR1 (LCDR1), light chain CDR2 (LCDR2), and light chain CDR3 (LCDR3), of a light chain variable region having an amino acid sequence as set forth in SEQ ID NO: 58 or 70;
the light chain CDR1 (LCDR1), light chain CDR2 (LCDR2), and light chain CDR3 (LCDR3), of a light chain variable region having an amino acid sequence as set forth in SEQ ID NO: 59 or 71; or
the light chain CDR1 (LCDR1), light chain CDR2 (LCDR2), and light chain CDR3 (LCDR3), of a light chain variable region having an amino acid sequence as set forth in SEQ ID NO: 64 or 72,
or a variant thereof wherein each of the LCDRs may contain one, two, or at most three amino acid variations.

11. The CD3 binding domain or the group of antigen-binding proteins according to claim 10, wherein the light chain variable region comprises an amino acid sequence as set forth in SEQ ID NO: 5; 41; 42; 43 or 68; 45 or 69; 49; 50; 52; 53; 56; 57; 58 or 70; 59 or 71; or 64 or 72, or having at least 80%, or at least 85%, or at least 90%, or at least 95%, sequence identity thereto.

12. The CD3 binding domain or the group of antigen-binding proteins according to claim 10 or 11, further comprising a CL region.

13. A binding moiety comprising a polypeptide as claimed in any one of claims 1-6, or a CD3 binding domain as claimed in claim 7 or 8.

14. The binding moiety according to claim 13, wherein the binding moiety is a multispecific binding moiety, in particular a bispecific or trispecific antibody.

15. A pharmaceutical composition comprising an effective amount of a polypeptide as claimed in any one of claims 1-6, or a CD3 binding domain as claimed in claim 7 or 8, or a binding moiety as claimed in claim 13 or 14, and a pharmaceutically acceptable carrier.

16. A polypeptide as claimed in any one of claims 1-6, or a CD3 binding domain as claimed in claim 7 or 8, or a binding moiety as claimed in claim 13 or 14, or a pharmaceutical composition as claimed in claim 15, for use in therapy.

17. A polypeptide as claimed in any one of claims 1-6, or a CD3 binding domain as claimed in claim 7 or 8, or a binding moiety as claimed in claim 13 or 14, or a pharmaceutical composition as claimed in claim 15, for use in the treatment of cancer.

18. A method for treating a disease, comprising administering an effective amount of a polypeptide as claimed in any one of claims 1-6, or a CD3 binding domain as claimed in claim 7 or 8, or a binding moiety as claimed in claim 13 or 14, or a pharmaceutical composition as claimed in claim 15, to an individual in need thereof.

19. A method for treating cancer, comprising administering an effective amount of a polypeptide as claimed in any one of claims 1-6, or a CD3 binding domain as claimed in claim 7 or 8, or a binding moiety as claimed in claim 13 or 14, or a pharmaceutical composition as claimed in claim 15, to an individual in need thereof.

20. A nucleic acid comprising a sequence encoding the polypeptide as claimed in any one of claims 1-6.

21. A vector comprising a nucleic acid as claimed in claim 20.

22. The vector according to claim 21, wherein the vector further comprises at least one nucleic acid sequence encoding a light chain variable region, and preferably a CL region.

23. The vector according to claim 22, wherein the light chain variable region is a light chain variable region comprising the light chain CDRs as defined in claim 10, or is a light chain variable region as defined in claim 11.

24. A cell comprising a nucleic acid as claimed in claim 20.

25. The cell according to claim 24, wherein the cell further comprises at least one nucleic acid comprising a sequence that encodes a light chain variable region, and preferably a CL region.

26. The cell according to claim 25, wherein the light chain variable region is a light chain variable region comprising the light chain CDRs as defined in claim 10, or is a light chain variable region as defined in claim 11.

27. A cell producing a polypeptide as claimed in any one of claims 1-6, or a CD3 binding domain as claimed in claim 7 or 8, or a binding moiety as claimed in claim 13 or 14.

28. The cell according to claim 27, wherein the cell is a recombinant cell comprising the vector as claimed in any one of claims 21-23.
